(19) United States
(12) Patent Application Publication
(10) Pub. No.: US 2003/0170763 A1
(43) Pub. Date: Sep. 11, 2003

(54) Ragweed allergens

(76) Inventors: Bob B. Buchanan, Berkeley, CA (US); Gregorio del Val, San Diego, CA (US); Oscar L. Frick, San Francisco, CA (US)

Correspondence Address: MORRISON & FOERSTER LLP, 425 MARKET STREET, SAN FRANCISCO, CA, 94105-2482, US

(21) Appl. No.: 10/067,484

(22) Filed: Feb. 4, 2002

Related U.S. Application Data
(60) Provisional application No. 60/266,686, filed on Feb. 5, 2001.

(51) Int. Cl. G01N 33/53; G01N 33/537; G01N 33/543; A61K 39/36; C07K 14/415

(52) U.S. Cl. 435/7.92; 424/185.1; 424/275.1; 530/379

(57) ABSTRACT

A 30 kDa ragweed complete pollen extract disulfide protein allergen has been purified from ragweed pollen. IgE immunoblots with sera of ragweed, walnut and ryegrass sensitive patients indicated that the 30 kDa protein is a major allergen. The 30 kDa protein finds use in allergy testing and immunotherapy regimens.

### RELATED APPLICATION

This application claims the benefit under 35 U.S.C. § 119(e) of U.S. provisional patent application No. 60/266,686, filed Feb. 05, 2001, which is hereby incorporated by reference in its entirety.

### FIELD OF THE INVENTION

The present invention relates to allergenic proteins from pollen of ragweed and fragments, derivatives and homologues thereof, and to allergenic proteins immunologically related thereto. More particularly, the present invention relates to a major allergenic 30 kDa disulfide protein isolated from complete ragweed pollen, an 8-10 kDa complete ragweed pollen extract disulfide protein, a 30 kDa defatted ragweed pollen extract disulfide protein and fragments, derivatives and homologues thereof.

### BACKGROUND OF THE INVENTION

Genetically predisposed individuals, who make up at least 10% of the population, become hypersensitized (allergic) to antigens from a variety of environmental sources to which they are exposed. Those antigens that can induce immediate and/or delayed types of hypersensitivity are known as allergens. Anaphylaxis or atopy, which includes the symptoms of hay fever, asthma, and hives, is one form of immediate allergy. It can be caused by a variety of atopic allergens, such as products of grasses, trees, weeds, animal dander, mites, insects, food, drugs and chemicals. Many individuals are allergic to ragweed pollen. In fact, ragweed is the major cause of pollen related allergies in much of the United States.

However, some of these ragweed sufferers do not test positively for allergic reactions in conventional tests suggesting that there may be as yet unidentified ragweed allergens.

There is thus an urgent need to identify additional ragweed allergens.

### SUMMARY OF THE INVENTION

In accordance with the present invention, it has been discovered that a number of ragweed proteins are only marginally extracted by prior art ragweed protein purification protocols. These proteins are, however, readily extracted by reversing the order of the extraction solutions—i.e., by applying the aqueous buffer first and then extracting this fraction with ether to remove interfering lipids. With this procedure, we have detected several novel ragweed pollen proteins. These proteins include a 30 kDa complete pollen extract disulfide glyco-protein also referred to as Ambt 7 herein, an 8-10 kDa complete pollen extract disulfide protein and a 30 kDa defatted pollen extract disulfide protein. Ambt 7 appears to be a major allergen: it has a high specificity for IgE from ragweed-sensitive patients and elicits a positive skin test in dogs sensitized to ragweed. This invention is directed to the isolation, purification and use of this glycoprotein, refererred herein as the “30 kDa ragweed pollen protein allergen” and “Ambt 7.” This invention is further directed to isolation, purification and use of an 8-10 kDa complete ragweed pollen extract disulfide protein and a 30 kDa defatted ragweed pollen extract disulfide protein

The present invention provides at least one purified 30 kDa ragweed complete pollen extract disulfide protein, at least one 8-10 kDa complete ragweed pollen extract disulfide protein and at least one 30 kDa defatted ragweed pollen extract disulfide protein or at least one antigenic fragment thereof, or derivative or homologue. A further aspect of the present invention provides an isolated antigenic fragment of an allergen from ragweed pollen, from a 30 kDa ragweed complete pollen extract disulfide protein allergen, from an 8-10 kDa complete ragweed pollen extract disulfide protein or from a 30 kDa defatted ragweed pollen extract disulfide protein.

The present invention is further directed to isolated peptides having the following peptide sequences:

| | |
|---|---|
| 1. | L/I L/I SGISNTVYANPK (SEQ ID NO: 1) |
| 2. | PTSFN L/I ATK (SEQ ID NO: 2) |
| 3. | L/I YGLVQFNR (SEQ ID NO: 3) |
| 4. | FY L/I FSTK (SEQ ID NO: 4) |
| 5. | FYATEV L/I D L/I D (SEQ ID NO: 5) |
| 6. | LLDNLHQQTPPDGFGR (SEQ ID NO: 6) |
| 7. | MYATEVLDLDGSK (SEQ ID NO: 7) |
| 8. | YSDGNFFGAGLDHQ (SEQ ID NO: 8) |
| 9. | LLNNMR (SEQ ID NO: 9) |
| 10. | VEASAELR (SEQ ID NO: 10) |
| 11. | LLSGLSDTV (SEQ ID NO: 11) |

The present invention is directed to a method of purifying a 30 kDa ragweed complete pollen extract disulfide protein allergen. The present invention is further directed to a method of purifying an 8-10 kDa complete ragweed pollen extract disulfide protein and a method of purifying a 30 kDa defatted ragweed pollen extract disulfide protein.

In one embodiment, the present invention is directed to the purification scheme depicted in FIG. 2B.

The present invention further provides purified nucleic acid sequences coding for the 30 kDa ragweed complete pollen extract disulfide protein allergen, the 8-10 kDa complete ragweed pollen extract disulfide protein and the 30 kDa defatted ragweed pollen extract disulfide protein or at least one antigenic fragment thereof, or derivative or homologue thereof, or the functional equivalent of the nucleic acid sequences. In particular, the present invention further provides purified nucleic acid sequences coding for peptides depicted in SEQ ID NO: 1-11. The present invention also provides expression vectors comprising a nucleic acid sequence coding for at least one 30 kDa ragweed complete pollen extract disulfide protein, one 8-10 kDa complete ragweed pollen extract disulfide protein and one 30 kDa defatted ragweed pollen extract disulfide protein or at least one antigenic fragment thereof, or derivative or homologue thereof, or the functional equivalent of the nucleic acid sequence. The present invention further provides host cells transformed to express a protein or peptide encoded by the nucleic acid sequences of the invention.

Still another aspect of the invention provides a modified ragweed pollen protein allergen which, when administered to a ragweed pollen-sensitive individual, reduces the allergic response of the individual to ragweed pollen. Preferably the ragweed pollen allergen is a modified 30 kDa ragweed complete pollen extract disulfide protein allergen, a modified 8-10 kDa complete ragweed pollen extract disulfide protein or a modified 30 kDa defatted ragweed pollen extract disulfide protein or derivative or homologue thereof. The present invention also provides at least one modified fragment of ragweed pollen protein allergen which, when administered to a ragweed pollen-sensitive individual, reduces the allergic response of the individual to ragweed pollen. Preferably the ragweed pollen protein allergen is a 30 kDa ragweed complete pollen extract disulfide protein, an 8-10 kDa complete ragweed pollen extract disulfide protein or a 30 kDa defatted ragweed pollen extract protein or antigenic fragment thereof, immunologically related to the 30 kDa ragweed complete pollen extract disulfide protein allergen, the 8-10 kDa complete ragweed pollen extract disulfide protein or the 30 kDa defatted ragweed pollen extract protein or fragment or derivative thereof is also provided by the present invention. The ragweed pollen protein allergen is generally in the form of a pharmaceutical composition.

In yet another aspect of the present invention, there is provided non-native (i.e., recombinant or chemically synthesized) 30 kDa ragweed pollen protein family members or their derivatives or homologues, or a non-native allergenic protein immunologically cross-reactive to antibodies to one or more 30 kDa ragweed complete pollen extract disulfide proteins, one or more 8-10 kDa complete ragweed pollen extract disulfide proteins or one or more 30 kDa defatted ragweed pollen extract protein family members or their derivatives or homologues. The present invention also provides purified native 30 kDa ragweed complete pollen disulfide protein allergens, purified native 8-10 kDa complete ragweed pollen extract disulfide protein allergens or purified native 30 kDa defatted ragweed pollen extract disulfide protein allergens or at least one fragment or derivative or homologue thereof.

Non-native 30 kDa ragweed complete pollen extract disulfide protein, non-native 8-10 kDa complete ragweed pollen extract disulfide protein or non-native 30 kDa defatted ragweed pollen extract disulfide protein and fragments or portions derived therefrom (peptides) can be used in methods of diagnosing, treating and preventing allergic reactions to ragweed pollen. Purified native 30 kDa ragweed complete pollen extract protein, purified native 8-10 kDa complete ragweed pollen extract disulfide protein or purified native 30 kDa defatted ragweed pollen extract protein fragments thereof, and homologues or derivatives thereof are also useful in methods of diagnosing, treating and preventing allergic reactions to ragweed pollen.

Still yet another aspect of the present invention relates to antibodies to non-native 30 kDa ragweed complete pollen extract disulfide protein, non-native 8-10 kDa complete ragweed pollen extract disulfide protein or non-native 30 kDa defatted ragweed pollen extract disulfide protein or derivatives or homologues thereof as well as antibodies raised against purified native 30 kDa ragweed complete pollen extract disulfide protein, purified native 8-10 kDa ragweed complete pollen extract disulfide protein or purified native 30 kDa defatted ragweed pollen extract protein or derivatives or homologues thereof.

The present invention is thus directed to an isolated protein having an amino acid sequence wherein the amino acid sequence is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

The present invention is further directed to a pharmaceutical composition including an isolated protein having an amino acid sequence wherein the amino acid sequence is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11. The pharmaceutical composition may be utilized in a method of treating pollen allergy in a mammal by administering a therapeutically effective amount of the protein to a mammal. The pharmaceutical composition may be also be utilized in a method of treating sensitivity to pollen in a mammal sensitive to pollen by administering to the mammal a therapeutically effective amount of the protein to a mammal. The mammal may be a human.

The present invention is further directed to a diagnostic composition for detecting pollen allergy wherein the diagnostic composition includes an isolated protein having an amino acid sequence wherein the amino acid sequence is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

In the invention, the pollen may be from any source. In one embodiment, the pollen is selected from walnut, ryegrass and ragweed pollen.

The present invention is further directed to an isolated nucleic acid having a nucleotide sequence encoding an amino acid sequence wherein the amino acid sequence is selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11. The nucleic acid of the invention may be in an expression vector. The expression vector may be in a host cell.

The present invention is further directed to an isolated pollen allergen substantially free of any other pollen proteins characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity. The allergen may be from any source. In a preferred embodiment, the allergen may be from walnut, ryegrass or ragweed pollen.

The present invention is further directed to a pharmaceutical composition having a pollen allergen substantially free of any other pollen proteins characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity. The allergen may be from any source. In a preferred embodiment, the allergen may be selected from walnut, ryegrass and ragweed pollen.

The present invention is further directed to a diagnostic composition for detecting allergic diseases which includes as the active ingredient a diagnostically effective amount of a pollen allergen substantially free of any other pollen proteins characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity. The allergen may be from any source. In a preferred embodiment, the allergen may be selected from walnut, ryegrass and ragweed pollen.

The present invention is further directed to a method of treating pollen allergy in a mammal by administering a pharmaceutically effective amount of an pollen allergen substantially free of any other pollen proteins characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity to a mammal, preferrably a human.

The present invention is further directed to a therapeutic composition having an isolated antigenic fragment of a ryegrass pollen allergen Ambt 7 wherein the antigenic fragment includes one or more amino acid sequences selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11 wherein the antigenic fragment has at least one epitope of the pollen allergen. The therapeutic composition generally includes a pharmaceutically effective carrier.

The epitope may be a T cell epitope or a B cell epitope.

The therapeutic composition may be administered to a mammal such as a human to treat sensitivity to ryegrass pollen.

The present invention is further directed to a therapeutic composition having an Ambt7 pollen allergen which is a polymorphic variant of a ryegrass Ambt7 pollen allergen wherein the polymorphic variant has an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11. The therapeutic composition may include a pharmaceutically acceptable carrier.

The therapeutic composition may be administered to a mammal in a method of treating sensitivity to ryegrass pollen.

The present invention is further directed to a kit for detecting Ambt7 pollen allergen wherein the kit includes one or more isolated proteins having an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11. The kit of may further include protein detection components such as antibodies. The kit may also further include directions for use of the kit.

The present invention is further directed to a method of purifying a pollen allergen, by: a) suspending the pollen in a liquid to form a pollen solution; b) centrifuging the pollen solution to produce a pollen protein supernatant; c) precipitating the protein in the pollen protein supernatant to form a protein precipitate; d) resuspending the protein precipitate in a protein precipitate buffer to form a resuspended protein mixture; e) extracting the resuspended protein mixture in organic solvent to form an aqueous phase and an organic phase; and f) purifying the pollen allergen from the aqueous phase.

In one embodiment of the method of purifying a pollen allergen, the protein in the pollen solution is precipitated with (NH₄)₂SO₄.

In another embodiment of the method of purifying a pollen allergen the organic solvent is petroleum ether.

In another embodiment of the method of purifying a pollen allergen, the pollen allergen is purified from the aqueous phase by chromatography or electrophoresis procedures. In the method, the chromatography procedure may be gel filtration or affinity chromatography.

The present invention is further directed to an isolated antibody that binds specifically to a protein comprising an amino acid sequence wherein the amino acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

The present invention is further directed to an isolated antibody that binds specifically to a pollen allergen substantially free of any other pollen proteins wherein the pollen allergen is characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity.

The antibodies of the invention may be a polyclonal or monclonal antibodies.

Further features of the present invention will be better understood from the following detailed description of the preferred embodiments of the invention in conjunction with the appended figures.

### BRIEF DESCRIPTION OF THE FIGURES

The invention will be better understood by reference to the Figures in which:

FIG. 1 shows the structure of the wall of ragweed pollen including: (1) the intine; (2) the exine; (3) the nexine; (4) the sexine; (5) the lipid layer; (6) the micropore; (7) the spine, (8) the protein; (9) cavity and (10) the protoplast.

FIG. 2a shows the prior art procedure for producing clinical pollen preparations.

FIG. 2b outlines a procedure for extracting the 30 kDa protein and other proteins from ragweed pollen.

FIG. 3 shows total protein, sulfhydryl protein and allergen profiles of aqueous extracts from complete and defatted ragweed pollen. Extracts were fractionated by Sephadex G50F chromatography and separated by SDS-PAGE (10-20%). FIG. 3A. shows gels stained for total protein. Gels were stained with Coomassie blue; each lane contained 3 to 30 μg protein. FIG. 3B shows sulfhydryl determination with monobromobimane. Sulfhydryl groups of proteins were labeled with monobromobimane and analyzed by UV light. FIG. 3C shows allergen determination by IgE immunoblotting. Proteins were transferred to nitrocellulose and probed with IgE of sera combined from 10 ragweed patients. The symbols depicted in FIG. 3c are: ( [symbol] ) 30 kDa protein, (*) 8-10 kDa protein (complete pollen) and (♦)second 30 kDa protein (defatted pollen).

FIG. 4 shows some properties of 30 kDa protein. SDS-PAGE (10-20%). FIG. 4A demonstrates that 30 kDa protein is glycosylated. The gel in FIG. 4A was stained for glycoprotein. Lane 1 contains soybean trypsin inhibitor (negative control, 5 μg); lane 2 contains 30 kDa protein (10 μg); and lane 3 contains horseradish peroxidase (positive control, 5 μg). FIG. 4B demonstrates that the 30 kDa protein contains at least one disulfide bond. The gel was examined under UV light following reaction with monobromobimane (mBBr). Ten μg protein was used.

FIG. 5 shows the response of sera from grass-sensitive patients to pollen preparations by SDS/PAGE (10-20%)/IgE immunoblotting. FIG. 5A shows the response of sera from 35 patients to pure 30 kDa protein. Patients showing binding to the 30 kDa protein are designated with a “+”. Those patients also showing a positive ImmunoCAP test are identified with an circle surrounding the “+”. Each lane on the gel contained 1.6 μg protein. FIG. 5B shows the response of sera from selected grass positive patients to commercial ragweed extract (left panel), complete pollen extract (middle panel) and purified 30 kDa protein (right panel). The commercial and complete extracts and the 30 kDa protein contained 25, 25 and 1.6 μg protein, respectively. In the control (C) treatment, sera and secondary antibody were omitted; the secondary antibody was omitted in lanes designated “Ab2.”

FIG. 6 shows the immunoblot inhibition of the 30 kDa protein with walnut and ryegrass pollen extracts and the demonstration of cross-reactivity. FIG. 6A is the control with no inhibitor protein added (C), ovalbumin as inhibitor protein (0, negative control), walnut (Juglans nigra) complete pollen extract (W) and ryegrass (Lolium perenne) complete pollen extract (R) added to sera prior to immunoblotting. In FIG. 6B the results with sera from an additional 17 patients positive to the 30 kDa protein are shown. In the control lanes (C) no allergen is added. In the lanes identified with an (R) ryegrass complete pollen extract is added as an inhibitor protein.

FIG. 7 shows the percentage of human IgE binding to known ragweed allergens vs. 30 kDa protein. An ELISA determination was carried out with sera from 10 ragweed-sensitive patients. 1 μg per ml protein was tested for each allergen. The values represent percent of total IgE bound by each allergen tested.

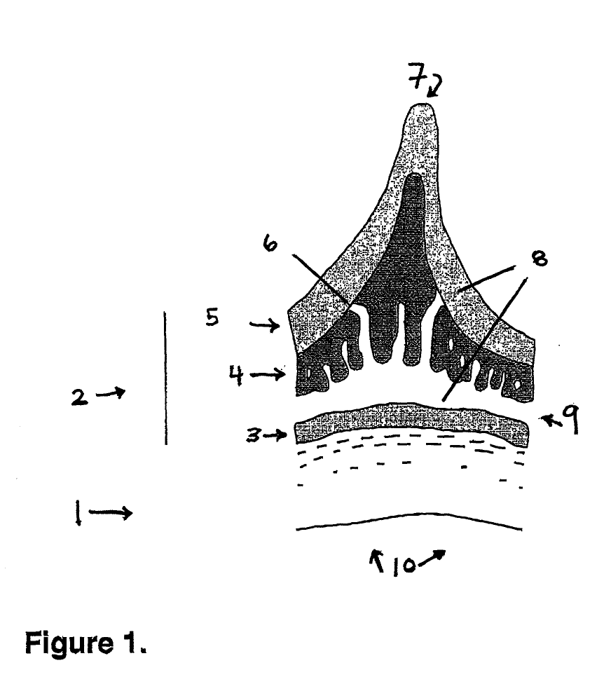

### DETAILED DESCRIPTION OF THE INVENTION

Giant ragweed allergen extract was purchased from Bayer, Inc. (Spokane, Wash.). Complete and defatted giant ragweed (Ambrosia trifida) pollen grains were purchased from Greer Laboratories (Lenoir, N.C.). These sources of pollen are not intended to limit the scope of the invention since they only represent one convenient supply of the pollen. The present invention can be practiced using ragweed pollen from any location or source.

In the following discussion, the 30 kDa ragweed complete pollen extract disulfide protein allergen is described and is identified as “the 30 kDa ragweed protein allergen” and as “Ambt 7.” This discussion is not limited to the 30 kDa ragweed protein allergen but applies equally to other ragweed protein allergens including the 8-10 kDa ragweed complete pollen extract disulfide protein, the 30 kDa ragweed defatted pollen extract disulfide protein and derivatives or homologues thereof.

Purification of Pollen Proteins

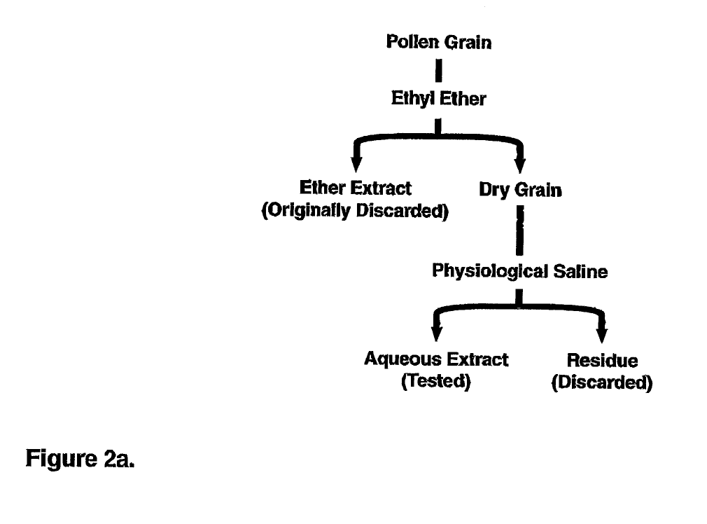

The present invention is directed to a method of purifying pollen proteins. The method finds particular use with ragweed pollen but is not limited to ragweed pollen. The method is outlined in FIG. 2b. Pollen is suspended in buffer such as 50 mM Tris-HCl, pH 7.4. One or more protease inhibitors such as phenylmethylsulfonyl fluoride and EDTA may be added to the buffer to reduce protein degradation. The suspended pollen is stirred gently at room temperature for a time sufficient to release pollen proteins, typically 30 minutes. The suspension is then centrifuged to precipitate the insoluble pollen material. The supernatant is then filtered. The proteins in the supernatant are precipitated with ammonium sulfate, for example at a 95% saturation. The floating pellet is then recovered by centrifugation and resuspended in buffer containing salt. In one embodiment, the buffer is 20 mM Tris-HCl (pH 7.5) and the salt is 200 mM NaCl. Lipids are then removed by extracting the resuspended protein pellet in an organic solvent such as petroleum ether. The mixture is then centrifuged, for example for 10 min at 48,000 g at 4° C., and the organic phase is discarded. The resulting clarified aqueous solution is filtered, for example, through a 0.2 uM filter. After filtration, the filtrate is separated on a gel filtration column to separate the various pollen proteins. After separation, the pollen proteins such as Ambt 7 can be further purified by procedures well known in the art such as SDS-PAGE, chromatography, etc.

The invention encompasses isolated or substantially purified nucleic acid or protein compositions. In the context of the present invention, an “isolated” or “purified” DNA molecule or an “isolated” or “purified” polypeptide is a DNA molecule or polypeptide that, by the hand of man, exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein of interest chemicals.

Gene(s) Encoding Ragweed Protein Allergens

“Gene,” is used, in respect of the present invention, in its broadest sense and refers to any contiguous sequence of nucleotides, the transcription of which leads to a mRNA molecule, which mRNA molecule is capable of being translated into a protein. The gene encoding a 30 kDa ragweed protein allergen family member means the nucleotide sequence encoding the protein or derivatives or homologues of the protein which may contain single or multiple amino acid substitutions, deletions or additions. A 30 kDa ragweed protein allergen gene also refers to cDNAs complementary to the mRNAs corresponding to the full or partial length of a 30 kDa protein.

It is expected that there are sequence polymorphisms in the nucleic acid sequence coding for each 30 kDa ragweed protein allergen family member, and it will be appreciated by one skilled in the art that one or more nucleotides in the nucleic acid sequence coding for a 30 kDa ragweed protein allergen family member may vary among individual ragweed plants due to natural allelic variation. Any and all such nucleotide variations and resulting amino acid polymorphisms are within the scope of the invention. It may also be appreciated by one skilled in the art that the 30 kDa ragweed protein allergen is a family of highly related genes whose proteins are present in ragweed pollen. Nucleotide sequences and corresponding deduced amino acid sequences of any and all such related family members including 30 kDa are within the scope of the invention.

Accordingly, it is within the scope of the present invention to encompass all proteins belonging to the 30 kDa ragweed protein allergen family, at least one fragment (peptide) of a 30 kDa ragweed protein allergen family member, and amino acid derivatives thereof, and to encompass nucleotide sequences, including DNA, cDNA and mRNA and homologue or degenerate forms thereof, encoding 30 kDa ragweed protein allergen family members or fragments thereof, or derivatives thereof. It is also within the scope of the invention to encompass purified native 30 kDa ragweed protein allergen, at least one fragment (peptide) thereof, and derivatives or homologues thereof. It is further in accordance with the present invention to include molecules such as polypeptides fused to a 30 kDa protein, or at least one fragment thereof, or derivatives thereof or to nucleotide sequences contiguous to such fragment and/or derivative-encoding nucleotide sequences.

For example, for some aspects of the present invention, it is desirable to produce a fusion protein comprising a 30 kDa ragweed protein allergen family member or at least one fragment thereof or their derivatives and an amino acid sequence from another peptide or protein, examples of the latter being enzymes such as beta-galactosidase, phosphatase, urease and fusion proteins incorporating purification moieties such as His-tags and the like. Most fusion proteins are formed by the expression of a recombinant gene in which two coding sequences have been joined together such that their reading frames are in phase. Alternatively, proteins or peptides can be linked in vitro by chemical means. All such fusion protein or hybrid genetic derivatives of a 30 kDa ragweed protein allergen or its encoding nucleotide sequences are encompassed by the present invention. Furthermore, by homologues and derivatives of a 30 kDa ragweed protein allergen is meant to include synthetic derivatives thereof. The nucleotide sequences encoding the 30 kDa ragweed protein allergen can be used to chemically synthesize the entire protein or generate any number of fragments (peptides) by chemical synthesis by well known methods (e.g., solid phase synthesis). All such chemically synthesized peptides are encompassed by the present invention. Accordingly, the present invention extends to isolated 30 kDa ragweed protein allergen family members, fragments thereof and their derivatives, homologues and immunological relatives made by recombinant means or by chemical synthesis.

The terms “isolated” and “purified” are used interchangeably herein and refer to peptides, protein, protein fragments, and nucleic acid sequences substantially free of cellular material or culture medium when produced by recombinant DNA techniques, or chemical precursors or other chemicals when synthesized chemically. The term “native purified” as used herein refers to proteins or fragments thereof purified from Ambrosia trifida pollen or other plant part. Furthermore, the present invention extends to proteins or fragments (peptides) corresponding in whole or part.

Fragments of nucleic acid within the scope of the invention include those coding for parts of the 30 kDa ragweed protein allergen that elicit an immune response in mammals, preferably humans, such as the stimulation of minimal amounts of IgE; binding of IgE; eliciting the production of IgG and IgM antibodies; or the eliciting of a T cell response such as proliferation and/or lymphokine secretion and/or the induction of T cell anergy. The foregoing fragments of the 30 kDa ragweed protein allergen are referred to herein as antigenic fragments. Fragments within the scope of the invention also include those capable of hybridizing with nucleic acid from other plant species for use in screening protocols to detect allergens that are cross-reactive with the 30 kDa ragweed protein allergen. As used herein, a fragment of the nucleic acid sequence coding for the 30 kDa ragweed protein allergen refers to a nucleotide sequence having fewer bases than the nucleotide sequence coding for the entire amino acid sequence of the 30 kDa ragweed protein allergen and/or a mature 30 kDa ragweed protein allergen family member. Generally, the nucleic acid sequence coding for the fragment or fragments of a the 30 kDa ragweed protein allergen family member will be selected from the bases coding for the mature 30 kDa ragweed protein allergen family member, however, in some instances it may be desirable to select all or a part of a fragment or fragments from the leader sequence portion of a nucleic acid sequence of the invention. A nucleic acid sequence of the invention may also contain linker sequences, restriction endonuclease sites and other sequences useful for cloning, expression or purification of the 30 kDa ragweed protein allergen or fragments thereof.

Antigenic Fragments of Ragweed Protein Allergens

Antigenic fragments of an allergen from ragweed pollen, preferably the 30 kDa ragweed protein allergen, may be obtained, for example, by screening peptides produced by recombinant methods from the corresponding fragment of the nucleic acid sequence of the invention coding for such peptides, synthesized chemically using techniques known in the art, or by degrading of the purified allergen. The peptide fragments of the protein allergen may be obtained by any method known in the art such as chemical cleavage of the allergen, arbitrary division of the allergen into fragments of a desired length with no overlap of the peptides, or preferably division of the allergen into overlapping fragments of a desired length. The fragments are tested to determine their antigenicity and allergenicity.

Fragments of recombinantly or synthetically produced 30 kDa ragweed protein allergen or of purified native 30 kDa ragweed protein allergen which are capable of eliciting a T cell response such as stimulation (i.e., proliferation or lymphokine secretion) and/or are capable of inducing T cell anergy are particularly desirable. Fragments of recombinantly or synthetically produced 30 kDa ragweed protein allergen or purified native 30 kDa ragweed protein allergen which do not bind immunoglobulin E (IgE) and/or which have minimal IgE stimulating activity are also desirable. If the fragment or fragments of a recombinantly or synthetically produced 30 kDa ragweed protein allergen family member or purified native 30 kDa ragweed protein allergen bind IgE, it is preferable that such binding does not lead to histamine release, e.g., such binding does not cause cross-linking of IgE on mast cells or basophils. Minimal IgE stimulating activity refers to IgE stimulating activity that is less than the amount of IgE production stimulated by whole recombinantly or synthetically produced 30 kDa ragweed protein allergen or whole purified native 30 kDa ragweed protein allergen.

Preferred fragments also include antigenic fragments which, when administered to a ragweed pollen-sensitive individual or an individual allergic to an allergen cross-reactive with ragweed pollen allergen, are capable of modifying the allergic response to ragweed pollen allergen of the individual, and antigenic fragments which, when administered to a ragweed pollen-sensitive individual, are capable of modifying B-cell response, T-cell response or both B-cell and T-cell response of the individual to a ragweed pollen allergen. As used herein modification of the allergic response of an individual sensitive to ragweed pollen allergen can be defined as non-responsiveness or diminution in symptoms to the allergen, as determined by standard clinical procedures (see e.g. Varney et al, British Medical Journal, (1990), 302:265-269), including diminution in grass pollen induced asthmatic symptoms (Suphioglu et al. (1992) Lancet 339: 569-572).

Antigenic fragments of the present invention which have T cell stimulating activity, and thus comprise at least one T cell epitope are particularly desirable. T cell epitopes are believed to be involved in initiation and perpetuation of the immune response to a protein allergen which is responsible for the clinical symptoms of allergy. These T cell epitopes are thought to trigger early events at the level of the T helper cell by binding to an appropriate HLA molecule on the surface of an antigen presenting cell and stimulating the relevant T cell subpopulation. These events lead to T cell proliferation, lymphokine secretion, local inflammatory reactions, recruitment of additional immune cells to the site, and activation of the B cell cascade leading to production of antibodies. One isotype of these antibodies, IgE, is fundamentally important to the development of allergic symptoms and its production is influenced early in the cascade of events, at the level of the T helper cell, by the nature of the lymphokines secreted. A T cell epitope is the basic element or smallest unit of recognition by a T cell receptor, where the epitope comprises amino acids essential to receptor recognition. Amino acid sequences which mimic those of the T cell epitopes and which modify the allergic response to protein allergens are within the scope of this invention.

Exposure of patients to purified protein allergens of the present invention or to the antigenic fragments of the present invention which comprise at least one T cell epitope and are derived from protein allergens may tolerize or anergize appropriate T cell subpopulations such that they become unresponsive to the protein allergen and do not participate in stimulating an immune response upon such exposure. In addition, administration of the protein allergen of the invention or an antigenic fragment of the present invention which comprises at least one T cell epitope may modify the lymphokine secretion profile as compared with exposure to the naturally-occurring protein allergen or portion thereof (e.g. result in a decrease of IL-4 and/or an increase in IL-2). Furthermore, exposure to such antigenic fragment or protein allergen may influence T cell subpopulations which normally participate in the response to the allergen such that these T cells are drawn away from the site(s) of normal exposure to the allergen (e.g., nasal mucosa, skin, and lung) towards the site(s) of therapeutic administration of the fragment or protein allergen. This redistribution of T cell subpopulations may ameliorate or reduce the ability of an individual's immune system to stimulate the usual immune response at the site of normal exposure to the allergen, resulting in a diminution in allergic symptoms.

Screening for IgE binding to the protein or fragments thereof may be performed by scratch tests or intradermal skin tests on laboratory animals or human volunteers, or in in vitro systems such as RAST (radioallergosorbent test), RAST inhibition, ELISA assay or radioimmunoassay (RIA).

Expression Vectors and Host Cells

The present invention provides expression vectors and host cells transformed to express the nucleic acid sequences encoding the ragweed protein allergen of the invention. Expression vectors of the invention comprise a nucleic acid sequence coding for at least one 30 kDa ragweed pollen allergen, or at least one antigenic fragment thereof, or derivative or homologue thereof, or the functional equivalent of such nucleic acid sequence. Nucleic acid sequences coding for 30 kDa ragweed protein allergen family members including 30 kDa ragweed protein allergen, or at least one fragment thereof may be expressed in prokaryotic or eukaryotic host cells. Suitable host cells include bacterial cells such as E. coli, insect cells, yeast, or mammalian cells such as Chinese hamster ovary cells (CHO). Suitable expression vectors, promoters, enhancers, and other expression control elements may be found in Sambrook et al. Molecular Cloning: A Laboratory Manual, second edition, Cold Spring Harbor Laboratory Press, Cold Spring Harbor, N.Y., 1989. Suitable vectors for expression in yeast include YepSecl (Baldari et al. (1987) Embo J. 6: 229-234); pMF (Kurjan and Herskowitz (1982) Cell 30: 933-943); and JRY88 (Schultz et al. (1987) Gene 54: 113-123).

For expression in E. coli, suitable expression vectors include pTRC (Amann et al. (1988) Gene 69: 301-315); pET-11d (Novagen, Madison, Wis.); PGEX (Amrad Corp., Melbourne, Australia); pMAL (N.E. Biolabs, Beverly, Mass.); pRIT5 (Pharmacia, Piscataway, N.J.); and PSEM (Knapp et al. (1990) BioTechniques 8: 280-281). The use of pTRC and pET-11d will lead to the expression of unfused protein. The use of pGEX, pMAL, pRIT5 and pSEM will lead to the expression of allergen fused to glutathione S-transferase (pGEX), maltose E binding protein (pMAL), protein A (pRIT5), or truncated β-galactosidase (PSEM). When a 30 kDa ragweed protein allergen family member, fragment, or fragments thereof is expressed as a fusion protein, it is particularly advantageous to introduce an enzymatic cleavage site at the fusion junction between the carrier protein and the 30 kDa protein family member or fragment thereof. A 30 kDa ragweed protein allergen family member or fragment thereof may then be recovered from the fusion protein through enzymatic cleavage at the enzymatic site and biochemical purification using conventional techniques for purification of proteins and peptides. Suitable enzymatic cleavage sites include those for blood clotting Factor Xa or thrombin for which the appropriate enzymes and protocols for cleavage are commercially available from for example Sigma Chemical Company, St. Louis, Mo. and N.E. Biolabs, Beverly, Mass.

Host cells can be transformed to express the nucleic acid sequences encoding the 30 kDa ragweed protein allergen of the invention using conventional techniques such as calcium phosphate or calcium chloride co-precipitation, DEAE-dextran-mediated transfection, or electroporation. Suitable methods for transforming the host cells may be found in Sambrook et al. supra, and other laboratory textbooks. The nucleic acid sequences of the invention may also be synthesized using standard techniques.

Production of Recombinant 30 kDa Ragweed Protein

Accordingly, another aspect of the present invention provides a method of producing recombinant 30 kDa ragweed protein allergen, or at least one fragment thereof, or their derivatives or homologues, or their immunological relatives (as hereinbefore defined) comprising culturing an organism containing a replicable recombinant DNA molecule, the molecule comprising a promoter capable of expression in the organism, a gene encoding a 30 kDa ragweed protein allergen family member, at least one fragment thereof, or homologue or derivative thereof, or immunological relatives thereof, located downstream of and transcribed from the promoter, a selectable marker and a DNA vehicle containing a prokaryotic or eukaryotic origin of replication, under conditions and for a time sufficient for the recombinant DNA molecule to be stably maintained and direct the synthesis of the 30 kDa ragweed protein allergen, at least one fragment thereof, or derivatives, homologues or immunological relatives thereof and then optionally isolating same.

30 kDa ragweed protein allergen and fragments (peptides) thereof can be purified from cell culture medium, host cells, or both using techniques known in the art for purifying peptides and proteins, including ion-exchange chromatography, hydrophobic chromatography, gel filtration chromatography, ultrafiltration, electrophoresis and immunopurification with antibodies specific for 30 kDa ragweed protein allergen or fragments thereof. The terms isolated and purified are used interchangeably herein and refer to peptides, protein, protein fragments, and nucleic acid sequences substantially free of cellular material or culture medium when produced by recombinant DNA techniques, or chemical precursors or other chemicals when synthesized chemically.

Another aspect of the invention provides protein preparations comprising isolated 30 kDa ragweed protein allergen or at least one fragment of 30 kDa ragweed protein allergen. In preferred embodiments of this aspect of the invention, the 30 kDa ragweed protein allergen or at least one fragment of the 30 kDa ragweed protein allergen is produced in a host cell transformed with a nucleic acid sequence coding for the protein or fragment.

Modifying an Individual's Allergic Response

It is possible to design peptides derived from the 30 kDa ragweed protein allergen which, when administered to a ragweed pollen sensitive individual in sufficient quantities, will modify the individual's allergic response to ragweed pollen. This can be done, for example, by examining the structure of 30 kDa ragweed protein allergen, producing peptides (via an expression system, synthetically or otherwise) to be examined for their ability to influence B-cell and/or T-cell responses in ragweed pollen sensitive individuals and selecting appropriate epitopes recognized by the cells. In referring to an epitope, the epitope will be the basic element or smallest unit of recognition by a receptor, particularly immunoglobulins, histocompatibility antigens and T cell receptors where the amino acids essential to the receptor recognition may be contiguous and/or non-contiguous in the amino acid sequence. Amino acid sequences which mimic those of the epitopes and which are capable of down regulating allergic response to ragweed pollen allergen can also be used.

It is now also possible to design an agent or a drug capable of blocking or inhibiting the ability of ragweed pollen allergen to induce an allergic reaction in ragweed pollen sensitive individuals. Such agents could be designed, for example, in such a manner that they would bind to relevant anti-30 kDa ragweed protein allergen-IgE's, thus preventing IgE-allergen binding and subsequent mast cell or basophil degranulation. Alternatively, such agents could bind to cellular components of the immune system, resulting in suppression or desensitization of the allergic response to Ambrosia trifida pollen allergens. A non-restrictive example of this is the use of appropriate B- and T-cell epitope peptides, or modifications thereof, based on the cDNA/protein structures of the present invention to suppress the allergic response to ragweed pollen. This can be carried out by defining the structures of B- and T-cell epitope peptides which affect B- and T-cell function in in vitro studies with blood components from ragweed pollen sensitive individuals.

Diagnosing Pollinosis

Protein, peptides or antibodies of the present invention can also be used for detecting and diagnosing ragweed pollinosis. For example, this could be done by combining blood or blood products obtained from an individual to be assessed for sensitivity to ragweed pollen with an isolated antigenic peptide or peptides of recombinantly or synthetically produced 30 kDa ragweed protein allergen or native purified 30 kDa ragweed protein allergen, under conditions appropriate for binding of components (e.g., antibodies, T-cells, B-cells) in the blood with the peptide(s) or protein and determining the extent to which such binding occurs. The extent to which binding occurs can be determined, for example, by assessing T cell function, T cell proliferation, B cell function, or binding of the protein, or fragment thereof, or derivative or homologue thereof to antibodies present in the blood or a combination thereof.

Additionally, sensitivity of a mammal to ragweed pollen may be determined by administering to a mammal a sufficient quantity of the 30 kDa ragweed pollen allergen, or at least one antigenic fragment thereof, or derivative or homologue thereof to provoke an allergic response in the mammal and determining the occurrence of an allergic response in the mammal to the ragweed pollen allergen. The ragweed pollen allergen(s), fragment(s) or derivative or homologue thereof used in this aspect of the present invention can be produced recombinantly or synthetically. Purified native 30 kDa ragweed protein allergen or fragments thereof may be substituted for a recombinantly or synthetically produced 30 kDa ragweed protein allergen or fragments thereof and used in the above method to determine sensitivity of the mammal to ragweed.

The invention further includes isolated allergenic proteins or fragments thereof that are immunologically related to 30 kDa ragweed protein allergen, including fragments, or derivatives or homologues thereof, such as by antibody cross-reactivity wherein the isolated allergenic proteins or fragments thereof are capable of binding to antibodies specific for the protein and peptides of the invention, or by T cell cross-reactivity wherein the isolated allergenic proteins or fragments thereof are capable of stimulating T cells specific for the protein and peptides of this invention.

Work by others has shown that high doses of allergens generally produce the best results (i.e., best symptom relief). However, many people are unable to tolerate large doses of allergens because of allergic reactions to the allergens. Modification of naturally-occurring allergens can be designed in such a manner that modified peptides or modified allergens which have the same or enhanced therapeutic properties as the corresponding naturally-occurring allergen but have reduced side effects (especially anaphylactic reactions) can be produced. These can be, for example, a protein or peptide of the present invention (e.g., one having all or a portion of the amino acid sequence of 30 kDa ragweed protein allergen, including purified native 30 kDa ragweed protein allergen), or a modified protein or peptide, or the protein or peptide analogue. It is possible to modify the structure of a protein or peptide of the invention for such purposes as increasing solubility, enhancing therapeutic or preventive efficacy or stability (e.g., shelf life ex vivo and resistance to proteolytic degradation in vivo. A modified protein or peptide can be produced in which the amino acid sequence has been altered, such as by amino acid substitution, deletion or addition, to modify immunogenicity and/or reduce allergenicity or to which a component has been added for the same purpose. A modified protein can further be produced by the use of thiol redox proteins to reduce protein intramolecular disulfide bonds as described in U.S. Pat. No. 6,114,504 which is hereby incorporated by reference in its entirety.

Treating Allergic Responses

Thus, the present invention provides modified ragweed pollen protein allergens which, when administered to a ragweed pollen-sensitive individual, reduce the allergic response of the individual to ragweed pollen. Preferred modified ragweed pollen protein allergens include modified 30 kDa ragweed pollen allergen or derivative or homologue thereof. The present invention also provides at least one modified fragment of ragweed pollen protein allergen which, when administered to a ragweed pollen-sensitive individual, reduces the allergic response of the individual to ragweed pollen. Preferably such modified fragments are at least one modified fragment of the 30 kDa ragweed pollen allergen or derivative or homologue thereof.

Another example of a modification of protein or peptides is substitution of cysteine residues preferably with alanine, serine, threonine, leucine or glutamic acid to minimize dimerization via disulfide linkages. Another example of modification of the peptides of the invention is by chemical modification of amino acid side chains or cyclization of the peptide.

In order to enhance stability and/or reactivity, the protein or peptides of the invention can also be modified to incorporate one or more polymorphisms in the amino acid sequence of the protein allergen resulting from natural allelic variation. Additionally, D-amino acids, non-natural amino acids or non-amino acid analogues can be substituted or added to produce a modified protein or peptide within the scope of this invention.

The purification of native 30 kDa ragweed pollen allergen is described in the examples herein.

Cloning of cDNAs

The DNA used in any embodiment of this invention can be cDNA obtained as described herein, or alternatively, can be any oligodeoxynucleotide sequence having all or a portion of a sequence represented herein, or their functional equivalents. Such oligodeoxynucleotide sequences can be produced chemically or mechanically, using known techniques.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller, 1988; the local homology algorithm of Smith et al. 1981; the homology alignment algorithm of Needleman and Wunsch 1970; the search-for-similarity-method of Pearson and Lipman 1988; the algorithm of Karlin and Altschul, 1990, modified as in Karlin and Altschul, 1993.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. 1988; Higgins et al. 1989; Corpet et al. 1988; Huang et al. 1992; and Pearson et al. 1994. The ALIGN program is based on the algorithm of Myers and Miller, supra. The BLAST programs of Altschul et al., 1990, are based on the algorithm of Karlin and Altschul supra.

Software for performing BLAST analyses is publicly available through the National Center for Biotechnology Information at the web site ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences (see, e.g., Karlin & Altschul (1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. 1997. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989). See the web site located at ncbi.nlm.nih.gov. Alignment may also be performed manually by inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%/, 71%, 72%, 73%, 74%, 75%, 76%, 77%, 78%, or 79%, preferably at least 80%, 81%, 82%, 83%, 84%, 85%, 86%, 87%, 88%, or 89%, more preferably at least 90%, 91%, 92%, 93%, or 94%, and most preferably at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, more preferably at least 80%, 90%, and most preferably at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, preferably 80%, 81%, 82%, 83%, 84%, 85%, 86%, 87%, 88%, or 89%, more preferably at least 90%, 91%, 92%, 93%, or 94%, or even more preferably, 95%, 96%, 97%, 98% or 99%, sequence identity to the reference sequence over a specified comparison window. Preferably, optimal alignment is conducted using the homology alignment algorithm of Needleman and Wunsch (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridization are sequence dependent, and are different under different environmental parameters. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, 1984; Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point I for the specific sequence and its complement at a defined ionic strength and pH. However, severely stringent conditions can utilize a hybridization and/or wash at 1, 2, 3, or 4° C. lower than the thermal melting point I; moderately stringent conditions can utilize a hybridization and/or wash at 6, 7, 8, 9, or 10° C. lower than the thermal melting point I; low stringency conditions can utilize a hybridization and/or wash at 11, 12, 13, 14, 15, or 20° C. lower than the thermal melting point I. Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. An extensive guide to the hybridization of nucleic acids is found in Tijssen, 1993. Generally, highly stringent hybridization and wash conditions are selected to be about 5° C. lower than the thermal melting point Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long robes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The following are examples of sets of hybridization/wash conditions that may be used to clone orthologous nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A 30 kDa ragweed protein allergen cDNA, or a portion thereof can be used to identify similar sequences in any variety or type of plant and thus, to identify or “pull out” sequences which have sufficient homology to hybridize to 30 kDa cDNA or mRNA or portion thereof, for example, DNA from allergens of other plants under conditions of low stringency. Those sequences which have sufficient homology (generally greater than 40%) can be selected for further assessment using the method described herein. Alternatively, high stringency conditions can be used as discussed above. In this manner, DNA of the present invention can be used to identify, in other types of plants, preferably related families, genera, or species, sequences encoding polypeptides having amino acid sequences similar to that of 30 kDa ragweed protein allergen and thus to identify allergens in other species. Thus, the present invention includes not only 30 kDa ragweed protein allergen, but also other allergens encoded by DNA which hybridizes, preferably under high stringency conditions, to DNA of the present invention.

The cloning of the cDNAs encoding the 30 kDa ragweed pollen allergen can be based on the recognition of the protein expressed by Escherichia coli transformed with lambda-gt 11 phage, using both specific monoclonal antibodies and specific serum IgE from grass pollen-sensitive patients.

The allergenic nature of the subject proteins are characterized in part, by their binding of the reaginic IgE antibodies which are present at high levels in sera of allergic patients. The IgE binding to the epitopes on allergic proteins can be tested in a chromogenic assay in which allergens immobilized on a solid support can be visualized by sequential incubation in (1) allergic patients serum; (2) enzyme-labeled anti-IgE antibodies.

A variety of expression vectors can be constructed for the production of the 30 kDa ragweed protein allergen, at least one fragment thereof or their derivatives. Thus, a further aspect of the present invention provides recombinant vectors comprising DNA sequences encoding the 30 kDa ragweed protein allergen, or derivatives or homologues thereof. More particularly, the present invention relates to recombinant DNA molecules comprising a eukaryotic or prokaryotic origin of replication, a detectable marker, DNA sequences encoding the 30 kDa ragweed protein allergen family members or derivatives or homologues thereof, or allergenic proteins cross-reactive with antibodies to the 30 kDa ragweed protein allergen or derivatives or homologues thereof, and, optionally, promoter sequences capable of directing transcription of 30 kDa ragweed protein allergen family members.

The 30 kDa ragweed protein allergen promoter is isolatable from ragweed genomic DNA by any number of procedures including use of promoter probes vectors, “chromosome walking” and S1 nuclease mapping and sequencing as DNA upstream of the transcription initiation site.

Accordingly, the present invention provides a recombinant DNA molecule comprising a ragweed pollen promoter sequence, and in particular a promoter for a gene encoding a 30 kDa ragweed protein allergen family member, or homologues or degenerate forms thereof located on the molecule and further having one or more restriction endonuclease sites downstream of the promoter such that a nucleotide sequence inserted into one or more of these sites is transcribable in the correct reading frame and is thereby a developmentally regulated, pollen-specific expression vector. As used herein, the “correct reading frame” has the same meaning as “in phase.” The aforementioned DNA molecule will preferably also have a selectable marker thereon, such as an antibiotic or other drug resistance gene, such as for example gene encoding resistance to ampicillin, carbenicillin, tetracycline, streptomycin and the like. The recombinant molecule will further comprise a means for stable inheritance in a prokaryotic and/or eukaryotic cell. This can be accomplished by the recombinant molecule carrying a eukaryotic and/or a prokaryotic origin of replication as hereinbefore described in relation to expression vectors.

Alternatively, the recombinant molecule will carry a means for integration into a host cell genome thereby permitting replication of the recombinant molecule in synchrony with the replication of said host cell genome. Examples of preferred prokaryotic hosts include cells E. coli, Bacillus and Pseudomonas amongst others. Preferred eukaryotic hosts include cells from yeast and fungi, insects, mammals and plants.

Antibodies to Ragweed Protein Allergens

The present invention extends to monoclonal and polyclonal antibodies to the 30 kDa ragweed protein allergen or at least one fragment of recombinantly or synthetically produced 30 kDa ragweed protein allergen or purified native 30 kDa ragweed protein allergen, produced according to methods well known to those of ordinary skill in the art.

Monoclonal Antibodies

The monoclonal antibodies may be used to screen the cDNA library for 30 kDa ragweed protein allergen clones to cross-reactivity with allergenic proteins from pollen of various related species. In the following discussion, reference to the 30 kDa ragweed protein allergen includes its derivatives, homologues and immunological relatives and chemical synthetic derivatives thereof. The following discussion also includes antibodies specific for purified 30 kDa ragweed protein allergen and fragments, derivative and homologues thereof. Such antibodies are contemplated to be useful in developing detection assays (immunoassays) for 30 kDa ragweed protein allergens especially during the monitoring of a therapeutic or diagnostic regimen and in the purification of recombinantly or synthetically produced 30 kDa ragweed protein allergen family members or purified native 30 kDa ragweed protein allergen. The antibodies may be monoclonal or polyclonal. Additionally, it is within the scope of this invention to include any second antibodies (monoclonal or polyclonal) directed to the first antibodies discussed above. The present invention further contemplates use of these first or second antibodies in detection assays and, for example, in monitoring the effect of a diagnostic or an administered pharmaceutical preparation. Furthermore, it is within the scope of the present invention to include antibodies to any molecules complexed with a 30 kDa ragweed protein allergen. Accordingly, an antibody to a 30 kDa ragweed protein allergen encompasses antibodies to such 30 kDa ragweed protein allergen, or antigenic parts thereof, and to any associated molecules (e.g., lipid regions, carrier molecules, fused proteins, and the like).

The 30 kDa ragweed protein allergen family members, or fragments thereof, considered herein are purified then utilized in antibody production. Both polyclonal and monoclonal antibodies are obtainable by immunization with recombinant, synthetic or native 30 kDa ragweed protein allergen family members, and either type is utilizable for immunoassays. The methods of obtaining both types of sera are well known in the art. Polyclonal sera are less preferred but are relatively easily prepared by injection of a suitable laboratory animal with an effective amount of a purified 30 kDa ragweed protein allergen family member, or antigenic parts thereof, collecting serum from the animal, and isolating specific sera by any of the known immunoabsorbent techniques. Although antibodies produced by this method are utilizable in virtually any type of immunoassay, they are generally less favored because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example, Kohler and Milstein (1975) Nature 256: 495-499, and Kohler and Milstein (1986) Eur. J. Immunol. 6: 511-519.

Unlike preparation of polyclonal sera, the choice of animal is dependent on the availability of appropriate immortal lines capable of fusing with lymphocytes. Mouse and rat have been the animals of choice in hybridoma technology and are preferably used. Humans can also be utilized as sources for sensitized lymphocytes if appropriate immortalized human (or nonhuman) cell lines are available. For the purpose of the present invention, the animal of choice may be injected with from about 0.1 mg to about 20 mg of purified recombinant or native 30 kDa ragweed protein allergen, or parts thereof. Usually the injecting material is emulsified in Freund's complete adjuvant. Boosting injections may also be required. The detection of antibody production can be carried out by testing the antisera with appropriately labeled antigen. Lymphocytes can be obtained by removing the spleen or lymph nodes of sensitized animals in a sterile fashion and carrying out fusion. Alternatively, lymphocytes can be stimulated or immunized in vitro, as described, for example, in Reading (1982) J. Immunol. Methods 53:261-291.

A number of cell lines suitable for fusion have been developed, and the choice of any particular line for hybridization protocols is directed by any one of a number of criteria such as speed, uniformity of growth characteristics, deficiency of its metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin.

Cell fusion can be induced either by virus, such as Epstein-Barr or Sendai virus, or polyethylene glycol. Polyethylene glycol (PEG) is the most efficacious agent for the fusion of mammalian somatic cells. PEG itself may be toxic for cells, and various concentrations should be tested for effects on viability before attempting fusion. The molecular weight range of PEG may be varied from 1000 to 6000. It gives best results when diluted to from about 20% to about 70% (w/v) in saline or serum-free medium. Exposure to PEG at 37° C. for about 30 seconds is preferred in the present case, utilizing murine cells. Extremes of temperature (i.e., about 45° C.) are avoided, and preincubation of each component of the fusion system at 37° C. prior to fusion can be useful. The ratio between lymphocytes and malignant cells is optimized to avoid cell fusion among spleen cells and a range of from about 1:1 to about 1:10 is commonly used.

The successfully fused cells can be separated from the myeloma line by any technique known by the art. The most common and preferred method is to chose a malignant line which is hypoxanthine guanine phosphoribosyl transferase (HGPRT) deficient, which will not grow in an aminopterin-containing medium used to allow only growth of hybrids, and aminopterin-containing medium used to allow only growth of hybrids and which is generally composed of hypoxanthine aminopterin, and thymidine, commonly known as the HAT medium. The fusion mixture can be grown in the HAT-containing culture medium immediately after the fusion or 24 hours later. The feeding schedules usually entail maintenance in HAT medium for two weeks and then feeding with either regular culture medium or hypoxanthine, thymidine-containing medium.

The growing colonies are then tested for the presence of antibodies that recognize the antigenic preparation. Detection of hybridoma antibodies can be performed using an assay where the antigen is bound to a solid support and allowed to react to hybridoma supernatants containing putative antibodies. The presence of antibodies may be detected by “sandwich” techniques using a variety of indicators. Most of the common methods are sufficiently sensitive for use in the range of antibody concentrations secreted during hybrid growth.

Cloning of hybrids can be carried out after 21-23 days of cell growth in selected medium. Cloning can be preformed by cell limiting dilution in fluid phase or by directly selecting single cells growing in semi-solid agarose. For limiting dilution, cell suspensions are diluted serially to yield a statistical probability of having only one cell per well. For the agarose technique, hybrids are seeded in a semisolid upper layer, over a lower layer containing feeder cells. The colonies from the upper layer may be picked up and eventually transferred to wells.

Antibody-secreting hybrids can be grown in various tissue culture flasks, yielding supernatants with variable concentrations of antibodies. In order to obtain higher concentrations, hybrids may be transferred into animals to obtain inflammatory ascites. Antibody-containing ascites can be harvested 8-12 days after intraperitoneal injection. The ascites contain a higher concentration of antibodies but include both monoclonals and immunoglobulins from the inflammatory ascites. Antibody purification may then be achieved by, for example, affinity chromatography.

Detection of the 30 kDa Ragweed Protein Allergen

The presence of 30 kDa ragweed protein allergen contemplated herein, or antibodies specific for same, in a patient's serum, plant or mammalian tissue or tissue extract, can be detected utilizing antibodies prepared as above, either monoclonal or polyclonal, in virtually any type of immunoassay. A wide range of immunoassay techniques are available as can be seen by reference to U.S. Pat. Nos. 4,015,043, 4,424,279 and 4,018,653 each of which is hereby incorporated by reference. This, of course, includes both single-site and two-site, or “sandwich”, assays of the non-competitive types, as well as in the traditional competitive binding assays. Sandwich assays are among the most useful and commonly used assays and are favored for use in the present invention. A number of variations of the sandwich assay technique exist, and all are intended to be encompassed by the present invention. Briefly, in a typical forward assay, an unlabeled antibody is immobilized in a solid substrate and the sample to be tested brought into contact with the bound molecule. After a suitable period of incubation, for a period of time sufficient to allow formation of an antibody-antigen secondary complex, a second antibody, labeled with a reporter molecule capable of producing a detectable signal is then added and incubated, allowing time sufficient for the formation of a tertiary complex of antibody-antigen-labeled antibody (e.g., antibody-30 kDa ragweed protein-antibody). Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labeled antibody are added simultaneously to the bound antibody, or a reverse assay in which the labeled antibody and sample to be tested are first combined, incubated and then added simultaneously to the bound antibody. These techniques are well known to those skilled in the art, including any minor variations as will be readily apparent.

In the typical forward sandwich assay, a first antibody having specificity for the 30 kDa ragweed protein allergen, or antigenic parts thereof, contemplated in this invention, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated at 25° C. for a period of time sufficient to allow binding of any subunit present in the antibody. The incubation period will vary but will generally be in the range of about 2-40 minutes. Following the incubation period, the antibody subunit solid phase is washed and dried and incubated with a second antibody specific for a portion of the hapten. The second antibody is linked to a reporter molecule which is used to indicate the binding of the second antibody to the hapten.

By “reporter molecule,” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e., radioisotopes). In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. For example, R-nitrophenyl phosphate is suitable for use with alkaline phosphatase conjugates; for peroxidase conjugates, 1,2-phenylenediamine, 5-aminosalicylic acid, or toluidine are commonly used. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the tertiary complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells or latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength, the fluorescein observed indicates the presence of the hapten of interest. Immunofluorescence and EIA techniques are both very well established in the art and are particularly preferred for the present method. However, other reporter molecules, such as radioisotope, chemilluminescent or bioluminescent molecules, may also be employed. It will be readily apparent to the skilled technician how to vary the procedure to suit the required purpose. It will also be apparent that the foregoing can be used to detect directly or indirectly (i.e., via antibodies) the 30 kDa ragweed protein allergen of this invention.

The term “protein chip” refers to chips for assaying proteins. Examples of protein chips include The Ciphergen ProteinChip® System available from Cipherphen which provides scientists with a versatile, integrated platform for biological research. Biologically important molecules from a variety of sources may be captured and analyzed on ProteinChip Arrays, using ProteinChip Readers and ProteinChip Software for rapid data analysis. The 30 kDa ragweed protein allergen of this invention may be analyzed using protein chips.

Another aspect of the present invention provides a method of detecting the 30 kDa ragweed protein allergen or a derivative or homologue thereof or an allergenic protein immunologically reactive with the 30 kDa ragweed protein allergen or derivatives or homologues present in serum, tissue extract, plant extract or other biological fluid comprising the steps of containing the serum, extract or fluid to be tested with an antibody to the 30 kDa ragweed protein allergen for a time and under conditions sufficient for an allergenic protein-antibody complex to form and subjecting the complex to a detecting means.

Kits

The present invention is also directed to a kit for the rapid and convenient assay for antibodies to the 30 kDa ragweed protein allergen or derivatives, homologues or immunological relatives thereof in mammalian body fluids (e.g., serum, tissue extracts, tissue fluids), in vitro cell culture supernatants, and cell lysates. The kit is compartmentalized to receive a first container adapted to an antigenic component thereof, and a second container adapted to contain an antibody to the 30 kDa ragweed protein allergen, the antibody being labelled with a reporter molecule capable of giving a detectable signal as hereinbefore described. If the reporter molecule is an enzyme, then a third container adapted to contain a substrate for the enzyme is provided. In one use of the subject kit, a sample to be tested is contacted with the contents of the first container for a time and under conditions for an antibody, if present in the sample, to bind to the 30 kDa ragweed protein allergen in the first container. If the 30 kDa ragweed protein allergen of the first container has bound to antibodies in the test fluid, the antibodies of the second container will bind to the secondary complex to form a tertiary complex and, since these antibodies are labeled with a reporter molecule, when subjected to a detecting means, the tertiary complex is detected.

Therefore, one aspect of the present invention is a kit for the detection of antibodies to a protein having allergenic properties, the protein from pollen of ragweed, the kit being compartmentalized to receive a first container adapted to contain recombinant 30 kDa ragweed protein allergen or its antigenic derivative or homologue or a purified native the 30 kDa ragweed protein allergen or its antigenic derivative or homologue, and a second container adapted to contain an antibody to the 30 kDa ragweed protein allergen or derivative or homologue thereof, the antibody labelled with a reporter molecule capable of giving a detectable signal. The “reporter molecule” may also involve agglutination of red blood cells (RBC) on latex beads. In this kit the reporter molecule is a radioisotope, an enzyme, a fluorescent molecule, a chemoilluminescent molecule, bioluminescent molecule or RBC. The kit alternatively comprises a container adapted to contain recombinant 30 kDa ragweed protein allergen or is antigenic derivative or homologue labeled with a reporter molecule capable of giving a detectable signal.

Immunotherapy

Because of the presence of allergens in the environment, hayfever and seasonal asthma continue to have significant morbidity and socio-economic impact on Western communities, despite advances made in their pharmacology and immunology. While the available spectrum of drugs, including anti-histamines and steroids have resulted in improvement in the treatment of allergic disease, they have unfortunate side-effects associated with long-term usage. Because of these problems, renewed interest has been shown in the immunotherapy of allergic disease. Immunotherapy involves the injection of potent allergen extracts to desensitize patents against allergic reactions (Bousquet, & Michel (1989) Allergy Clin. Immunol. News 1: 7-10). Unfortunately, the pollen preparations used as allergens are polyvalent and of poor quality. Consequently, concentrations used are frequently high in order to induce IgG responses, but may be lethal through triggering of systemic reactions, including anaphylaxis. The cloned gene product or synthetic peptides based on the sequence of allergens provides a safer medium for therapy since it can be quality controlled, characterized and standardized.

The precise mechanism for symptomatic relief remains hypothetical. However, administration of a preparation comprising recombinant, synthetic or purified native 30 kDa ragweed protein allergen or at least one antigenic fragment thereof, of the instant invention to a ragweed sensitive individual will modify the allergic response of a ragweed sensitive individual to ragweed pollen allergens, e.g. by modifying the B-cell response to 30 kDa ragweed protein allergen, the T-cell response to 30 kDa ragweed protein allergen, or both the B cell and T cell response to 30 kDa ragweed protein allergen.

Accordingly, the present invention provides a method for desensitizing a human allergic to ragweed pollens which comprises administering a desensitizing-effective amount of 30 kDa ragweed protein allergen or at least one fragment or a derivative, homologue, or immunological relative thereof, for a time and under conditions sufficient to effect desensitization of the human to the grass pollen.

The present invention also provides a method of treating sensitivity to ragweed pollen in a mammal sensitive to such pollen, comprising administering to the mammal a therapeutically effective amount of a therapeutic composition of the invention. The present invention further provides a method of treating sensitivity to ragweed pollen allergen or an allergen immunologically cross-reactive with ragweed pollen allergen comprising administering to a mammal a therapeutically effective amount of the protein preparation of the invention.

Through the use of the peptides and protein of the present invention, preparations of consistent, well-defined composition and biological activity can be made and administered for therapeutic purposes (e.g., to modify the allergic response of a ragweed pollen sensitive individual to pollen of such plants. Administration of such peptides or protein may, for example, modify B-cell response to 30 kDa ragweed protein allergen, T-cell response to 30 kDa ragweed protein allergen, or both responses. Purified peptides can also be used to study the mechanism of immunotherapy of ragweed protein allergy and to design modified derivatives or analogues useful in immunotherapy.

Pharmaceutical Compositions

The present invention, therefore, provides a pharmaceutical composition comprising a desensitizing or therapeutically effective amount of 30 kDa ragweed protein allergens or derivatives, homologues or immunological relatives thereof and one or more pharmaceutically acceptable carriers and/or diluents. The active ingredients of a pharmaceutical composition comprising 30 kDa ragweed protein allergens is contemplated to exhibit excellent therapeutic activity, for example, in the desensitization of humans allergic to ragweed pollen when administered in amount which depends on the particular case. For example, from about 0.5 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intranasal, intradermal or suppository routes or implanting (e.g., using slow release molecules). Depending on the route of administration, the active ingredients which comprise the pharmaceutical composition of the invention may be required to be coated in a material to protect the ingredients from the action of enzymes, acids and other natural conditions which may inactivate said ingredients. For example, the 30 kDa ragweed protein allergens may be administered in an adjuvant, co-administered with enzyme inhibitors or in liposomes. Adjuvant is used in its broadest sense and includes any immune stimulating compound, such as interferon. Adjuvants contemplated herein include resorcinols, non-ionic surfactants such as polyoxyethylene oleyl ether and n-hexadecyl polyethylene ether. Enzyme inhibitors include pancreatic trypsin. Liposomes include water-in-oil-in-water CGF emulsions as well as conventional liposomes. For purposes of inducing T cell anergy, the pharmaceutical composition if preferably administered in non-immunogenic form (e.g. it does not contain adjuvant).

The active compounds may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders of the extemporaneous dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When at least one 30 kDa ragweed protein allergen family member, or at least one fragment thereof is suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be carried and may conveniently be between about 5 to 80% of the weight of the unit. The amount of active compound in such therapeutically useful compositions is such that a suitable dosage will be obtained. Preferred compositions or preparations according to the present invention are prepared so that an oral dosage unit form contains between about 10 and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (1) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from about 10 μg to about 2000 mg. Expressed in proportions, the active compound is generally present in from about 10 μg to about 2000 mg/ml of carrier. In the case of compositions containing supplementary active ingredients, the dosages are determined by reference to the usual dose and manner of administration of the ingredients.

The present invention is further illustrated by the following non-limiting Example.

### EXAMPLE

A. Materials and Methods

Pollen grains. Complete and defatted Giant ragweed (Ambrosia trifida) pollen grains were purchased from Greer laboratories (Lenoir, N.C.). A control pollen extract from giant ragweed was purchased from Bayer, Inc (Spokane Wash.) and also used for skin tests in dogs; a pollen extract mixture of giant, short and Western ragweed was purchased from Bayer, Inc. for clinical percutaneous skin tests in humans.

Protein quantification and amino acid sequencing. Protein was quantified with the Bradford assay using gamma globulin as standard. Bradford, M. A. (1976) A rapid and sensitive method for the quantification of microgram quantities of protein utilizing the principle of protein-dye binding. Analytical Biochemistry 72: 248-254. Amino acid sequences were determined with tryptic peptides by mass spectroscopy by the Protein Structure Laboratory, University of California, Davis.

Protein labeling with monobromobimane (mBBr). Protein solutions were reduced with 1 mM dithiothreitol at 100° C., 5 min. The sample was cooled to room temperature and labeled with 0.2 mM mBBr by incubating 20 min at room temperature. The reaction was stopped by adding 10 mM beta-mercaptoethanol and the proteins were precipitated by adding trichloroacetic acid to 12%. After washing with 100% acetone, the pellet was subjected to SDS-PAGE and the extent of protein labeling was visualized by spectroscopy at 365 nm as described by Wong et al. Wong, J. H., Kobrehel, K. and Buchanan, B. B. (1995) Thioredoxin and seed proteins. Methods in Enzymology 252: 228-240.

Glycoprotein staining. After separation by SDS-PAGE, proteins were stained for glycosylation with the in gel GelCode Glycoprotein Staining kit from Pierce (Rockford, Ill.).

Gel electrophoresis. Samples were reduced by 1 mM dithiothreitol at 100° C., 5 min. and after cooling to room temperature were separated in 10-20% SDS-PAGE. Laemmli, U. K. (1970) Cleaveage of structural proteins during the assembly of the head of bacteriophage T4. Nature 227: 680-5. After the run, gels were fixed, stained with Coomassie brilliant blue G-250 and destained in 10% acid acetic. We observed that low molecular weight proteins such as Amb t 5 required reduction by dithiothreitol to be stained effectively with Coomassie blue and that the use of methanol for destaining removes them from the gel.

Immunoblots. Proteins were transferred from 10-20% SDS-PAGE to a nitrocellulose membrane under semi-dry conditions with a 20% methanol solution (25 mM Tris base, 192 mM glycine, 0.1% SDS) for 1 h at 4° C. Nitrocellulose membranes were briefly stained with Ponceau Red to verify the extent of transfer and then blocked by incubating twice with a 3% cow's milk solution (20 mM Tris-HCl, pH 7.5, 150 mM NaCl and 0.2% Triton X-100) for 30 min at room temperature. Membranes were then incubated in 1 to 10 dilution of sera in the same solution overnight at 4° C. Finally, membranes were incubated at room temperature for 1 h in a 1000-X dilution of secondary anti-human IgE conjugated to horseradish peroxidase (Sigma) and reactive protein identified with 3,3′,5,5′-tetramethylbenzidine (TMB) substrate kit for peroxidase from Vector laboratories (Burlingame, Calif.).

ELISA. Microplates were coated with 100 μl of each purified allergen (see below) at 1 μg per ml in 10 mM PBS, pH 7.5, by leaving overnight at 4° C. The plates were washed 3 times with 10 mM PBS, pH 7.2, containing 0.05% Triton X-100 (Buffer A), coated a second time with 1% milk in Buffer A for 2 hr at 37° C. and washed as before. Serial dilutions (10 -50-X) of each of 10 different human sera were added in Buffer A and incubation was continued for an additional 2 hr at 37° C. Plates were washed as before and incubated with a 1000-X dilution of secondary anti-human IgE conjugated to peroxidase (Sigma) for 2 hr at 37° C. TMB substrate for measuring the conjugated peroxidase was added according to the manufacturer's instructions and the reaction was monitored for linearity at 650 nm for 1 h using a microplate reader. The reaction was stopped with 50 μl 0.1 N H₂SO₄ and absorbance was measured at 450 nm. The experiment was repeated 3 times and the mean was calculated for each allergen with the 10 human sera tested.

Skin tests. Procedures to measure the type I hypersensitivity reaction by skin tests with sensitized dogs have been described elsewhere. Ermel, R. W., Kock, M., Griffey, S. M., Reinhart, G. A. and Frick, O. L. (1997) The atopic dog: a model for food allergy. Laboratory Animal Science 47: 40-9. In brief, 0.5% Evans blue dye (0.2 ml/kg) was injected intravenously 5 minutes prior to skin testing. Aliquots of 0.1 ml of the test protein solution in half log dilutions were injected intradermally on ventral abdominal skin. Skin tests were read by the same experienced blinded observer scoring the two perpendicular diameters of each blue spot. Appropriate negative controls (diluted in PBS) were included for each animal tested.

Human sera. Seven sera from patients with fall pollinosis and a positive prick skin test to a mixture of giant, short and Western ragweed pollen extracts (wheal 3 mm greater than the negative control) and 15 sera from patients with a positive Pharmacia ImmunoCAP specific IgE assay to giant ragweed (IU/kl>0.35) were used. Many of these subjects were known to have resided in the Midwest, East or Southeast areas of the United States prior to California residence, but full geographic history was not available on all subjects. An additional 20 sera from patients known to be sensitive to perennial ryegrass (Lolium perenne) (by prick skin testing, positive ImmunoCAP and late spring allergic rhinitis) but negative on ImmunoCAP assay to giant ragweed were also included.

Calculation of Relative allergenicity. Equal amounts of protein, either purified or in pollen extracts, were injected and assigned a relative value indicating the minimal quantity producing a wheal: 330 ng protein=1, 100 ng=2, 33 ng=3, 10 ng=4, 3.3 ng=5, 1 ng=6 and 0.33 ng=7. We then summed the values for each purified protein or extract for the two groups of dogs tested [4 old (7-year-old) and 5 young (2-year-old) dogs.]

Protein extraction and allergen purification. An adaptation of the method of Marsh et al. 1981 was followed. Marsh, D. G., Belin, L., Bruce, A., Lichtenstein, L. M. and Hussain, R. (1981) Rapidly released allergens from short ragweed pollen. I. Kinetics of release of known allergens in relation to biologic activity. Journal of Allergy and Clinical Immunology 67 (3): 206-16. Hussain, R., Norman, P. S. and Marsh, D. G. (1981) Rapidly released allergens from short ragweed pollen. II. Identification and partial purification. Journal of Allergy and Clinical Immunology 67 (3): 217-22. To work out the procedure, an extract was prepared from 10 g of pollen (complete or defatted) and subjected to different exploration treatments. For the purification of proteins, 100 g of complete pollen was used. In brief, the pollen was suspended at 1 g to 10 ml of cold buffer [50 mM Tris-HCl pH 7.4 containing 1 μM phenylmethylsulfonyl fluoride (PMSF) and 1 mM EDTA-Na] and stirred gently for 30 min at room temperature. The mixture was centrifuged for 10 min, 25,900×g, 4° C. The pellet, containing the pollen grains, was set aside and the supernatant fraction was recentrifuged and filtered through Whatman quantitative filters. Ammonium sulfate was added to 95% saturation yielding a floating pellet that was recovered by centrifugation (10 min, 25,900×g, 4° C.) and resuspended to a minimal volume with 20 mM Tris-HCl pH 7.5, containing 200 mM NaCl. With both complete and defatted pollen, the high quantity of lipids was removed by extraction with an equal volume of petroleum ether. The mixture was centrifuged (10 min, 48,400×g, 4° C.) and the organic fraction was discarded. The petroleum ether step was repeated at least 4 times. The resulting clarified aqueous solution was filtered through a 0.2 μM filter and, in the case of 100 g pollen, separated on a Sephadex G-50F gel filtration column (2.1×90 cm) equilibrated and eluted with the same buffer used to dissolve the sample. The fractions were analyzed by 10-20% SDS-PAGE, combined according to protein size and dialyzed against 10 mM K-phosphate buffer pH 7.0 overnight at 4° C. The remainder of the procedure is described for 100 g pollen as starting material.

30 kDa protein. The combined Sephadex G-50F fractions of the proteins from complete pollen were dialyzed against 10 mM K-phosphate pH 7.0 and applied first to a 6 ml Resource S column and then to a 6 ml Resource Q column, both equilibrated with 20 mM K-phosphate pH 7.0. The 30 kDa protein (known herein as the 30 kDa ragweed protein allergen and the 30 kDa ragweed complete pollen extract disulfide protein allergen) was not retained in either case and was recovered in the column pass-through fractions. A considerable amount of contaminants was retained on the columns and thus removed from the 30 kDa protein. The fractions containing the 30 kDa protein were subjected to precipitation with ammonium sulfate, 95% saturation, and centrifuged for 10 min, 48,400×g, 4° C. The supernatant fraction was discarded and the pellet was resuspended in 2-3 ml volume of 50 mM K-phosphate, pH 7.0, containing 2.0 M ammonium sulfate. The fraction was applied to a 1 ml Resource Isopropyl column equilibrated with the same buffer. The 30 kDa protein was eluted at 1.7 M using a 60 ml gradient ranging from 2 to 0 M ammonium sulfate. The fractions containing the 30 kDa protein were localized by SDS-PAGE (using mBBr labeling and Coomassie blue staining), combined and dialyzed against 5 mM K-phosphate, pH 7.0. Sodium-acetate pH 4.75 was then added to 30 mM and the sample was applied to a 6 ml Resource S equilibrated with the same buffer. The 30 kDa protein was eluted at 100-200 mM NaCl in a 120 ml gradient ranging from 0 to 300 mM NaCl. The fractions were neutralized by adding 50 mM K-phosphate pH 7.0, dialyzed against 10 mM of the same buffer, concentrated by ultrafiltration with a YM-10 Amicon membrane and stored at −70° C. Protein was quantified using the Bradford assay.

30 kDa protein (alternate procedure). Following addition of 20 mM Tris-HCl pH 7.5, 1 mM MgCl2, 1 mM CaCl2, 0.5 M NaCl, the combined Sephadex G-50F fractions were applied to a 18 ml Concanavalin A affinity column (Sigma Chemical Co., St. Louis, Mo.) equilibrated with the same buffer. The 30 kDa protein was retained and eluted with a solution of 20 mM Tris-HCl pH 7.5, 0.5 M NaCl and 0.5 M methyl-alpha-D-glucopyranoside. The fractions containing the 30 kDa protein were combined and dialyzed against 5 mM K-phosphate buffer pH 7.0 using a membrane with a 25,000 M.W. cutoff pore. Finally, the protein was applied to a 6 ml Resource S column equilibrated with 20 mM Na-acetate, pH 6.0, and was recovered in the pass-through fractions.

Amb t 5. Ammonium sulfate was added to 2.6 M to the low molecular weight Sephadex G-50F fractions from complete pollen containing Amb t 5. The resulting solution was fractionated on a 1 ml HiTrap Phenyl Sepharose column equilibrated with 200 mM phosphate buffer, pH 7.0, and eluted with a 50 ml of ammonium sulfate gradient ranging from 2.5 to 0 M in this same buffer. Pure Amb t 5 was recovered in a single peak at approximately 0.8 M ammonium sulfate, dialyzed against 5 mM K-phosphate buffer, pH 7.0, and stored at −70° C. for further experiments. Protein was quantified using a molar coefficient of extinction at 278 nm of 5800. Gill, S. C. and von Hippel, P. H. (1989) Calculations of protein extinction coefficients from amino acid sequence data. Analytical Biochemistry 182: 319-26.

Amb t 3 and cytochrome c. The maximal yield of Amb t 3 was obtained from complete pollen and cytochrome c from defatted pollen. The Sephadex G-50F fractions containing proteins of about 10-20 kDa from the respective pollen preparations were combined and applied to a 6 ml Resource S column equilibrated with 20 mM K-phosphate buffer, pH 7.0. Amb t 3 and cytochrome c were separated with a 120 ml gradient from 0 to 500 mM NaCl in 20 mM K-phosphate buffer pH 7.0. Amb t 3 was eluted at 100-120 mM and cytochrome c at 150-170 mM NaCl. The presence of Amb t 3 was confirmed by adding a crystal of potassium ferricyanide to the fractions to oxidize the copper of Amb t 3, thereby turning the solution blue. Fractions containing Amb t 3 were combined and made 2 M with ammonium sulfate. The final purification of Amb t 3 and cytochrome c was achieved by separation through a 1 ml HiTrap Phenyl Sepharose column equilibrated with 200 mM K-phosphate buffer, pH 7.0. The column was eluted with a 60 ml ammonium sulfate gradient ranging from (1) 1.75 to 0 M for Amb t 3, which was eluted at 1.4 M, and (2) 2.0 to 0 M for cytochrome c, eluted at 1.2 M. The purified proteins were then dialyzed against 10 mM K-phosphate buffer, pH 7.0 and stored in aliquots at −70° C. Protein content was quantified using the Bradford assay and, in the case of Amb t 3, using a molar coefficient of extinction at 278 nm of 26600. Gill, S. C. and von Hippel, P. H. (1989) Calculations of protein extinction coefficients from amino acid sequence data. Analytical Biochemistry 182: 319-26.

Amb t 1 and 2. The largest quantity of Amb t 1-2 was obtained from defatted pollen. The Sephadex G-50F fractions containing proteins of 35 kDa and greater were dialyzed against 20 mM Tris-HCl, pH 7.9, containing 14 mM beta-mercaptoethanol. The following steps were modified from the procedure of King's group. King, T. P. (1972) Separation of proteins by ammonium sulfate gradient solubilization. Biochemistry 11: 367-371. Ishizaka, K., Kishimoto, T. Delespesse, G. and King, T. P. (1974) Immunogenic properties of modified antigen E. I. Presence of specific determinants of T cells in denaturated antigen and polypeptide chains. Journal of Immunology 113: 70-7. King, T. P. Philip, S. N. and Tao, N. (1874) Chemical modifications of the major allergen of ragweed pollen, antigen E. Immunochemistry 11:83-92. Ishizaka, K. Okudaira, H. and King T. P. (1975) Immunogenic properties of modified antigen E. II. Ability of urea-denatured antigen and alpha-polypeptide chain to prime cells specific for antigen E. Journal of Immunology 114:110-5. King, T. P., Kouchmian, L., Ishizaka, K., Lichtenstein, L. and Norman, P. S. (1975) Immunochemical studies of dextran coupled ragweed pollen allergen, antigen E. Archives of Biochemistry and Biophysics 169: 464-473. The protein solution was applied to a 6 ml Resource Q column equilibrated with 20 mM Tris-HCl, pH 7.9, and eluted with a gradient of 240 ml ranging from 0 to 500 mM NaCl. As described earlier by King's group, the proteins were eluted at about 50 mM NaCl. Ammonium sulfate was added to 2.5 M and the solution was applied to a 1 ml Resource Isopropyl column equilibrated with 100 mM K-phosphate, pH 7.0, containing 2.5 M ammonium sulfate. Amb t 1 and Amb t 2 were eluted at about 1.4 M ammonium sulfate in a 100 ml gradient ranging from 2.5 M to 0 M. The positive fractions were identified as above, combined, concentrated by ultrafiltration through a YM-30 Amicon membrane, dialyzed against 10 mM K-phosphate pH 7.0 and stored in aliquots at −70° C. The positive fractions were shown to have the correct molecular mass by SDS-PAGE; allergenicity was confirmed by skin tests with ragweed-sensitive dogs. Protein was quantified with the Bradford assay.

Immunoblot inhibitions to investigate possible cross-reactivity of other pollens with the 30 kDa protein were performed for 4 patient sera from patients who lived in ragweed endemic areas prior to relocation to California. Complete pollens of perennial ryegrass (Lolium perenne) and black walnut (Juglans nigra) were purchased from Hollister-Stier (Spokane, Wash.). In brief, 5 g of pollen were extracted in PBS (1:20 w:v) overnight at 4° C. and pelleted by centrifugation as described above. After that, the supernatant was defatted with ether and the organic phase was discarded. Preincubation of sera with 250 μg/ml perennial ryegrass (Lolium perenne) pollen extract, black walnut (Juglans nigra) pollen extract, or ovalbumin (Sigma) as a negative control was done overnight at 4° C. The sera were then incubated with nitrocellulose strips as above, washed, and ¹²⁵I-labeled anti-IgE (Hycor Biomedical, Inc., Garden Grove, Calif.) was used as the secondary antibody for immunoblotting as described by Teuber et al., 1999.

B. Results and Discussion

In a comparative allergen study, Marsh and his colleagues Marsh, et al. (1981), Hussain, et al. (1981) found no significant differences between complete and defatted ragweed pollen. By contrast, our preliminary investigation on the hypersensitivity response of atopics dogs suggested a difference in the allergen profile with complete and defatted pollen Ermel, et al. (1997) and G. del Val, et al., J. Allergy Clin. Immunol. 103, 690 (1999). The results seemed of interest because of possible relevance to the allergenic response to pollen. As demonstrated by Bridger and Protcor, Ann. Otol. Rhinol. Laryngol. 80, 445 (1971), albumin beads of the pollen grain size remain in the nose and larynx for about a half-hour prior to being swallowed. A large quantity of pollen protein is, therefore, released during that time. Howlett, et al. J. Cellsci, 13,603 (1973). We have, therefore, focused on the proteins released within the first 20 minutes of extraction, thinking that new allergens could be present in this fraction (the “first released proteins”). This fraction is known to contain several allergens, Amb t 5, Amb t 3 and cytochrome c) Marsh, et al. (1981), Hussain, et al. (1981). The major allergen (Amb t 1), however, requires several hours for maximal release King (1972), Ishizaka, et al. (1974), King et al. (1974) and Ishizaka, et al. (1974). Our initial results suggested that defatted pollen differs from its complete counterpart by a deficiency in the first released protein allergens (data not shown).

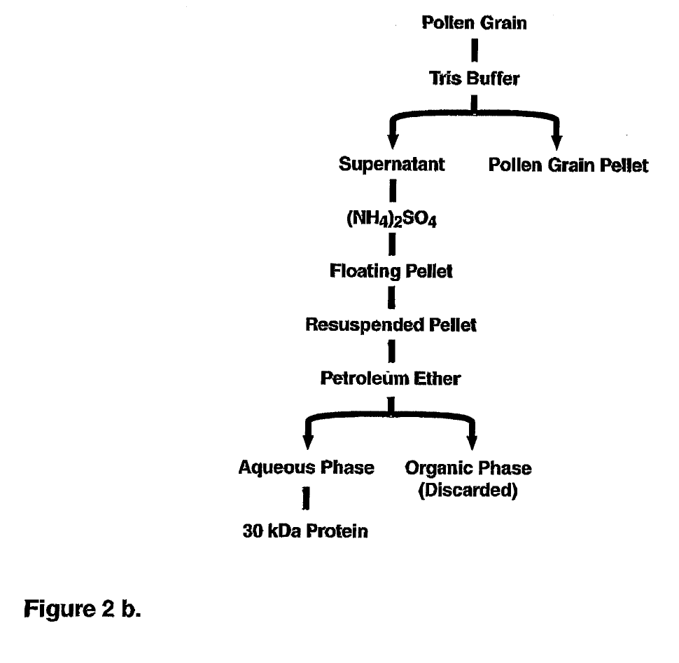

Identification of the 30 kDa protein as an allergen. This finding prompted us to carry out an analysis of the allergens present in the first released proteins of complete and defatted ragweed pollen Bradford (1976). Owing to the large amount of lipid recovered in aqueous extracts of complete pollen grain, we devised a procedure to obtain large quantities of “first released proteins”. In the current comparative study, the aqueous solutions obtained after the petroleum ether extraction and filtration steps were applied to a Sephadex G-50F gel filtration column and the fractions probed with sera from ragweed-sensitive patients. The fractions from the two types of pollen preparations were then examined with respect to (a) total protein using Coomassie blue stain (FIG. 3A); (b) proteins containing sulfhydryl groups using a fluorescent probe, monobromobimane (mBBr), applied after reduction with dithiothreitol (9, 24-27) (FIG. 3B); and (c) allergens using pooled sera from 10 patients with specific IgE directed against giant ragweed (FIG. 3C). S. S. Teuberm, K. C. Jarvis, A. M. Dandekar, W. R. Peterson, A. A. Ansari, J. Allergy Clin. Immunol. 104, 1311(1999).

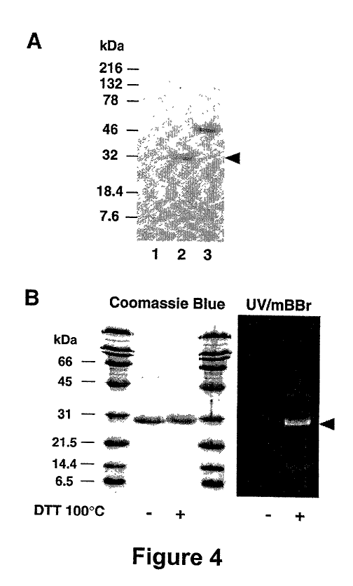

Significant differences were noted between the complete and defatted pollens. Standing out was a 30 kDa protein (identified with a wedge) that contained a sulfhydryl component and was delayed in elution from the gel filtration column such that it was recovered with the low molecular weight proteins, e.g., Amb t 5 (FIGS. 3A and 3B). The 30 kDa protein was recognized by IgE in the pool of human sera used (FIG. 3C). By exposing the gel filtration fractions to individual sera, we found that the 30 kDa protein was recognized by sera of all ten patients tested, whereas the other allergens were not (data not shown). This finding suggested that the 30 kDa protein was a major allergen (see FIGS. 5A and 5B below).

In addition to the 30 kDa protein, several proteins not previously described were found to bind human IgE. These include (i) an 8-10 kDa disulfide protein (G-50F fraction #36) in complete pollen extract, just below Amb t 3 (the 8-10 kDa protein is identified with a star in FIG. 3C), and (ii) a second 30 kDa protein in the defatted extract that was not delayed by the gel filtration (G-50F fraction #16, identified with a diamond in FIG. 3C). Finally, as Marsh and collaborators reported, we found, that unlike Amb t 3 and Amb t 5, the level of the major allergen Amb t 1-2 was low in the protein fraction from complete pollen. A significantly higher quantity of Amb t 1-2, was, however, found in the corresponding fraction from defatted pollen.

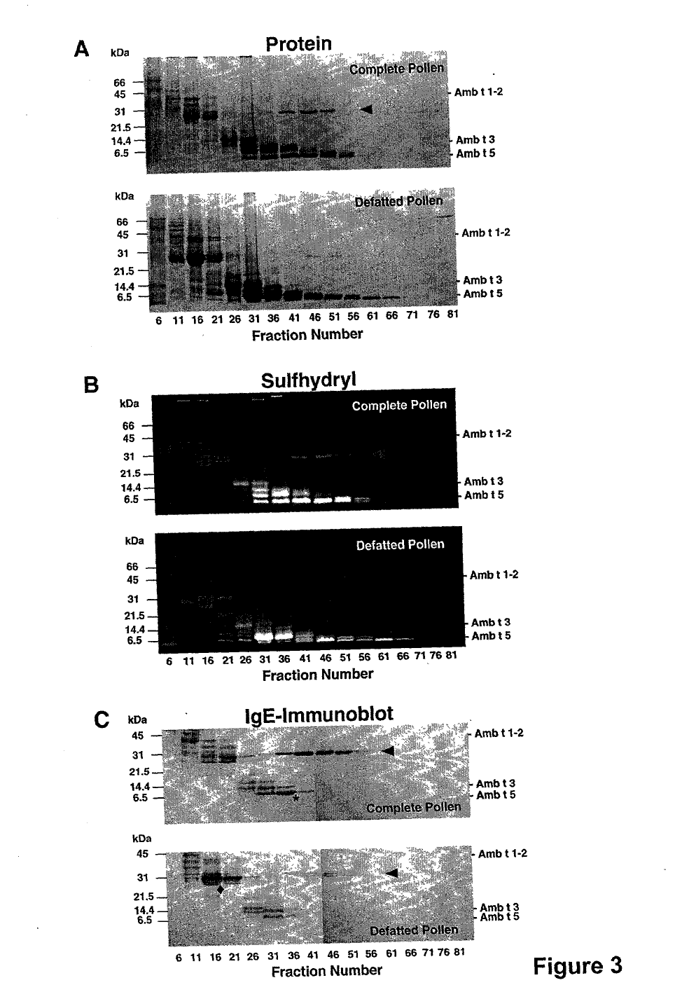

Properties of the 30 kDa protein. Because of its apparent allergenic properties, we purified the 30 kDa protein from complete pollen to homogeneity as detailed above. In characterizing the protein, we found it to have properties of many known allergens R. D. J. Huby, R. J. Dearman, I. Kimber, Toxicol Sci. 55, 235 (2000). S. B. Lehrer, W. E. Horner, G. Reese, Crit. Rev. Food Sci. Nutr.. 553-64 (1996). D. D. Metcalfe et al., Crit. Rev. Food Sci. Nutr. 36, S165 (1996). J. D. Astwood, J. N. Leach, R. L. Fuchs, Nat. Biotechnol. 14, 1269 (1996); i.e., it (i) was a glycoprotein (FIG. 4A), (ii) had at least one disulfide bond (FIG. 4B) and (iii) had a pI about 8.0 (determined by isoelectrofocusing electrophoresis, data not shown). The finding of a glycan moiety in the protein led us to another interesting property. After the gel filtration separation, the 30 kDa protein was strongly retained (>90%) on a glycoprotein affinity column, concanavalin A. This feature simplifies the purification of the protein to a few steps. Further experiments indicated that the 30 kDa protein was also retained at a lower level by lectin affinity columns (data not shown). The affinity data suggest that the glycan moiety is composed mainly of alpha-D-mannose and alpha-D-glucose.

Importance of the 30 kDa protein as a human allergen. The next query was to assess the allergenic importance of the 30 kDa protein with ragweed-sensitive patients. An allergen is qualified as being major if recognized immunologically by at least 50% of a minimum of 15 sensitive patients S. B. Lehrer, et al. (1966). In our case, we initially screened sera from 7 individuals who had a history of ragweed pollinosis (allergic rhinitis), positive prick skin tests to a mixture of giant, short, and Western ragweed pollen extracts and were positive in an approved in vitro (Pharmacia ImmunoCAP FEIA) test for IgE to giant ragweed (kU/l>0.35). All seven sera showed IgE binding to the 30 kDa protein. To pursue our study, we blindly analyzed 35 additional sera from patients identified as being allergic to grass and possibly ragweed. Among them, 31 patient sera showed binding to the 30 kDa protein (identified with a “+” in FIG. 5A). Fifteen of these patient sera had a positive ImmunoCAP to ragweed (kU/l>0.35) and also showed IgE binding to the 30 kDa protein (identified with a circle and a “+” in FIG. 5A). Hence, 22 patient sera positive to ragweed by skin test and/or ImmunoCAP (7 from first group, 15 from grass group) showed IgE binding to the 30 kDa protein, qualifying it as a major allergen (FIG. 5A). Of the remaining 20 grass allergic control sera, 16 identified with a “+” in FIG. 3A showed IgE binding to the 30 kDa protein, some very faintly, but were negative on ImmunoCAP to ragweed. To investigate this point further, immunoblotting was performed against the first released protein fraction from complete pollen containing the 30 kDa protein and against a commercial counterpart. Twenty-two sera were strongly positive to several proteins in the complete pollen extract, whereas only 18 were positive to a commercial ragweed preparation (positive on ImmunoCAP testing). Immunoblots showing sera from 6 patients selected from the group positive to complete pollen extract are shown in FIG. 5B. Interestingly, 4 of 22 patient sera (patient nos. 7, 9, 18 and 26) negative with the ImmunoCAP assay for giant ragweed showed IgE binding to the complete pollen extract and the 30 kDa allergen, but not to the commercial counterpart (no. 7 is shown in FIG. 5B). Two patient sera (nos. 4 and 7) that were barely positive (0.37 kU/l) or negative on ImmunoCAP were negative when tested with the commercial extract, but were positive with the first released proteins of complete pollen as well as with the 30 kDa allergen (FIG. 5B).

Briefly, of the 42 patient sera tested we found: 22 positive by the Pharmacia ImmunoCAP assay or skin tests, 29 positive to first released proteins of complete ragweed pollen extract, and 39 positive to the purified 30 kDa protein.

To summarize, the IgE immunoblots using sera from patients with a positive ImmunoCAP or positive skin tests to ragweed indicate that the 30 kDa protein is a major allergen. Furthermore, our study suggests that use of commercial defatted extracts may give false negatives. That is, we identified patients whose sera were positive to complete pollen extract, but were negative by the ImmunoCAP assay. Thus, there were at least 4 strong reactors to complete pollen extract (patient sera nos. 7, 9, 18 and 26) who failed to react with the ImmunoCap screen (FIG. 5B for no. 7, other data not shown). Based on this finding, the ImmunoCap assay may miss about 18% of ragweed-sensitive patients (4 of the 22 patients whose sera IgE bound proteins in the complete pollen extract). However, this point would need to be correlated clinically by challenge testing in those negative by ImmunoCAP (defatted pollen is used in its preparation) but positive to complete pollen. In addition, we identified 16 out of the 20 grass allergic sera that were reactive to the 30 kDa protein (sera from patients nos. 1, 6, 7, 8, 9, 12, 17, 18, 22, 23, 24, 25, 26, 30, 31 and 34) and were not detected with the other tests. The clinical history in regard to ragweed sensitivity was not known for these patients.

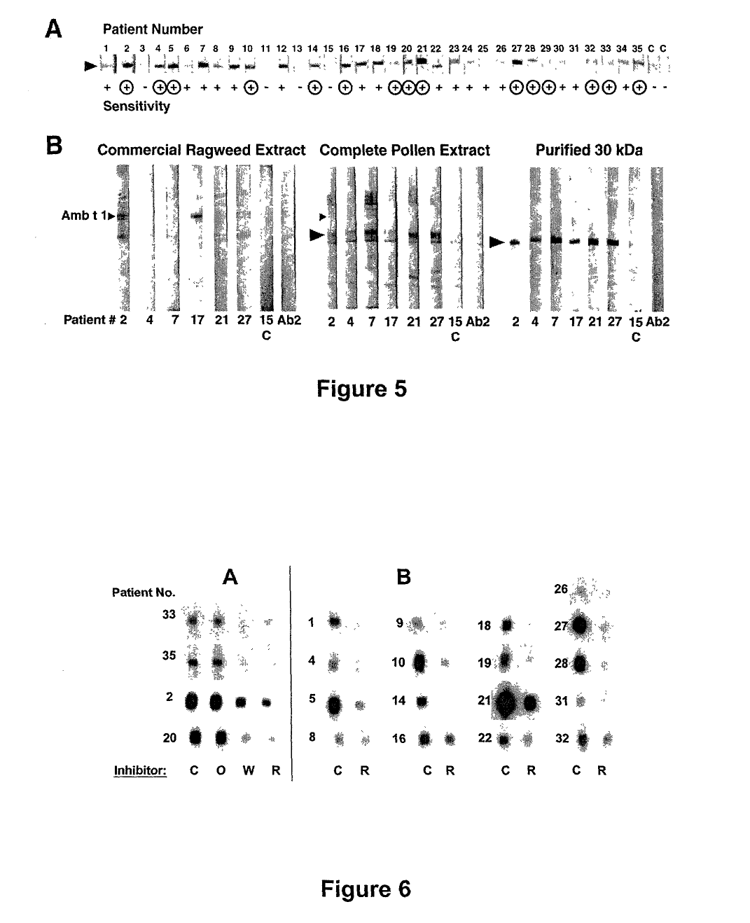

The finding that 80% (16 of 20) of grass allergic patient sera (negative to ragweed by ImmunoCAP) showed binding to the 30 kDa protein raised the possibility that this protein cross-reacts with a counterpart protein in other allergen sources. To test this possibility, immunoblot inhibition was performed using two sera that strongly (nos. 2, 20) and two that weakly bound the 30 kDa protein (nos. 33, 35) from patients in the group of 35 grass allergic patients whose clinical and geographic history supported a diagnosis of ragweed pollinosis. FIG. 6A (left panel) shows that while the control protein ovalbumin (O) was inactive and resembled the control treatment without added inhibitor (C). By contrast, treatment of sera with extracts from complete pollens of black walnut (W) and perennial ryegrass (R) partially or totally inhibited IgE binding to the 30 kDa protein. An additional 17 patient sera were screened for cross-reactivity between the 30 kDa protein and ryegrass pollen extract. In each case, ryegrass extract either partially or totally absorbed IgE directed to the 30 kDa protein (FIG. 6B right panel). These results suggest that the 30 kDa protein from ragweed pollen cross reacts with a counterpart in other pollens (walnut and rye grass).

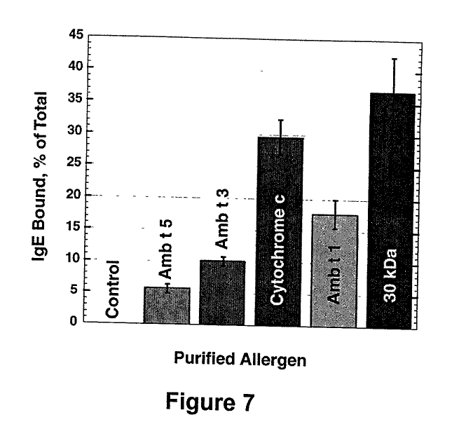

A comparative assessment of allergenicity using an ELISA protocol (38) with sera from 10 ragweed-sensitive patients (same as for FIG. 3C), confirmed the immunoblot results in identifying the 30 kDa protein as an allergen (FIG. 7). Moreover, the data indicated that the 30 kDa protein bound a higher percentage of human IgE from these patients than any of the known allergens tested (39,40) including Amb t 1, the allergen proposed to be the strongest in ragweed (t test, p=0.02). The results provide further evidence that the 30 kDa protein is a major allergen in ragweed pollen.

A remaining question was whether the allergenicity of the 30 kDa protein could be detected in vivo. To this end, we tested the purified protein with an atopic dog colony Ermel, et al. (1997) sensitized to giant ragweed pollen and observed a hypersensitive response (41). We obtained positive results with 16 of the 19 animals tested (Table 1). The old dogs were more sensitive to the 30 kDa protein (by 10-fold) than their young counterparts. In addition, whereas the old dogs were uniformly sensitive to the new allergen, 20% of the young dogs were not. These results indicate that the low level of 30 kDa protein present in commercial preparations is sufficient to sensitize dogs if injected repeatedly over an extended period. This observation also supports the conclusion that the defatted commercial extract is deficient in major allergens.

Table 1: Average of minimum amount of 30 kDa protein from complete ragweed pollen producing a wheal.

$$\frac{\begin{matrix}Old Dogs , n = {5}^{*} & 1 ng \\ Young Dogs , n = {14}^{**} & 10 ng\end{matrix}}{\begin{matrix}{}^{*} 5 dogs positive \\ {}^{**} 11 dogs positive\end{matrix}}$$

The next question was to determine whether the 30 kDa protein had been described. We therefore obtained partial amino acid sequences by mass spectrometry. The results indicated that aside from marginal similarity to envelop glycoproteins, the 30 kDa protein had not been previously described from pollen or other sources.

Amino acid sequence of tryptic peptidase

| | |
|---|---|
| 1. | L/I L/I SGISNTVYANPK (SEQ ID NO: 1) |
| 2. | PTSFN L/I ATK (SEQ ID NO: 2) |
| 3. | L/I YGLVQFNR (SEQ ID NO: 3) |
| 4. | FY L/I FSTK (SEQ ID NO: 4) |
| 5. | FYATEV L/I D L/I D*(SEQ ID NO: 5) |
| 6. | LLDNLHQQTPPDGFGR (SEQ ID NO: 6) |
| 7. | MYATEVLDLDGSK (SEQ ID NO: 7) |
| 8. | YSDGNFFGAGLDHQ (SEQ ID NO: 8) |
| 9. | LLNNMR (SEQ ID NO: 9) |
| 10. | VEASAELR (SEQ ID NO: 10) |
| 11. | LLSGLSDTV (SEQ ID NO: 11) |

*Homology with envelope glycoproteins

We have developed a simple procedure to extract allergens associated with the extracellular lipid layer of pollen. The procedure has yielded a major new allergen, a 30 kDa protein, that is released from complete ragweed pollen within minutes and is discarded in the commercial defatting process. The protein is glycosylated, has a molecular mass of 30 kDa, is water soluble and has at least one disulfide bond. Sequence data are forthcoming. The finding of the 30 kDa protein, as well as other uncharacterized allergens among proteins released in minutes from ragweed pollen grains, suggests that these allergens are in close association with the appearance of the first allergic symptoms to pollen. We propose to name the new allergen Amb t 7 (submitted to the WHO/IUIS Allergen Nomenclature Sub-Committee).

The data at hand indicate that up to 18% of patients sensitive to ragweed pollen by IgE immunoblotting are not diagnosed owing to deficiency of the Amb t 7 protein and possibly other allergens in current clinical preparations used for skin testing and in vitro specific IgE assays. Allergists encounter occasional patients who show typical seasonal variation in allergic rhinitis symptoms, but who are negative on prick skin testing to the usual allergen panels. Recognition that aqueous extracts from complete pollen contain an allergen such as the 30 kDa protein may be useful in producing improved formulas for diagnosing these patients and for immunotherapy. Skin test studies may be utilized to compare patients having known ragweed allergy with immunotolerant counterparts in order to correlate reactivity of Amb t 7 with the classical ragweed allergy syndrome.

We claim:

1. An isolated protein comprising an amino acid sequence wherein said amino acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

2. A pharmaceutical composition comprising the protein of claim 1.

3. A diagnostic composition for detecting pollen allergy comprising the protein of claim 1.

4. The diagnostic composition of claim 3 wherein said pollen is ragweed pollen.

5. A method of treating pollen allergy in a mammal comprising administering a pharmaceutically effective amount of the protein of claim 1 to said mammal.

6. The method of claim 5 wherein said pollen is ragweed pollen.

7. The method of claim 5 wherein said mammal is a human.

8. A method of treating sensitivity to pollen in a mammal sensitive to pollen comprising administering to said mammal a therapeutically effective amount of the protein of claim 1.

9. The method of claim 8 wherein wherein said pollen is ragweed pollen.

10. The method of claim 8 wherein said mammal is a human.

11. An isolated nucleic acid compromising a nucleotide sequence encoding an amino acid sequence wherein said amino acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

12. An expression vector comprising a nucleic acid of claim 11.

13. A host cell comprising an expression vector of claim 11.

14. An isolated pollen allergen substantially free of any other pollen proteins characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity.

15. The allergen of claim 14 wherein said pollen is ragweed pollen.

16. A pharmaceutical composition comprising the allergen of claim 14.

17. A diagnostic composition for detecting allergic diseases which comprises as the active ingredient a diagnostically effective amount of the allergen of claim 14.

18. The diagnostic composition of claim 17 wherein said allergen is ragweed pollen.

19. A method of treating pollen allergy in a mammal comprising administering a pharmaceutically effective amount of the allergen of claim 14 to said mammal.

20. The method of claim 19 wherein said mammal is a human.

21. The method of claim 19 wherein said pollen allergy is ragweed pollen allergy.

22. A therapeutic composition comprising an antigenic fragment of a ragweed pollen allergen Ambt 7 wherein said antigenic fragment comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11 wherein said antigenic fragment comprises at least one epitope of said pollen allergen and a pharmaceutically effective carrier.

23. The therapeutic composition of claim 22 wherein said epitope is a T cell epitope.

24. The therapeutic composition of claim 22 wherein said epitope is a B cell epitope.

25. A method of treating pollen sensitivity in a mammal comprising administering a therapeutically effective amount of the therapeutic composition of claim 22 to a mammal.

26. The method of claim 25 wherein said mammal is a human.

27. The method of claim 25 wherein said pollen sensitivity is ragweed pollen sensitivity.

28. A therapeutic composition comprising an Ambt7 pollen allergen which is a polymorphic variant of a ragweed Ambt7 pollen allergen wherein said polymorphic variant comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11 and a pharmaceutically acceptable carrier.

29. A method of treating pollen sensitivity in a mammal comprising administering a therapeutically effective amount of the therapeutic composition of claim 28 to a mammal.

30. The method of claim 29 wherein said mammal is a human.

31. The method of claim 29 wherein said pollen sensitivity is ragweed pollen sensitivity.

32. A kit for detecting Ambt7 pollen allergen comprising one or more proteins wherein said one or more proteins comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6₁ SEQ ID NO: 7, SEQ ID NO: 8, SEQ ID NO: 9, SEQ ID NO: 10, and SEQ ID NO: 11.

33. The kit of claim 32 further including protein detection components.

34. The kit of claim 32 wherein said protein detection components include antibodies.

35. The kit of claim 32 further including directions for use of the kit.

36. A method of purifying a pollen allergen, comprising:
a) suspending said pollen in a liquid to form a pollen solution;
b) centrifuging said pollen solution to produce a pollen protein supernatant;
c) precipitating said protein in said pollen protein supernatant to form a protein precipitate;
d) resuspending said protein precipitate in a protein precipitate buffer to form a resuspended protein mixture;
e) extracting said resuspended protein mixture in organic solvent to form an aqueous phase and an organic phase; and
f) purifying said pollen allergen from said aqueous phase.

37. The method of claim 36 wherein protein in said pollen solution is precipitated with (NH₄)₂ SO₄.

38. The method of claim 36 wherein said organic solvent is petroleum ether.

39. The method of claim 36 wherein said pollen allergen is purified from said aqueous phase by chromatography or electrophoresis procedures.

40. The method of claim 39 wherein said chromatography procedure is gel filtration or affinity chromatography.

41. An isolated antibody that binds specifically to a protein comprising an amino acid sequence wherein said amino acid sequence is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

42. The antibody of claim 41 wherein said antibody is a polyclonal antibody.

43. The antibody of claim 41 wherein said antibody is a monclonal antibody.

44. An isolated antibody that binds specifically to a pollen allergen substantially free of any other pollen proteins wherein said pollen allergen is characterized by the following physiochemical and biological properties: a) being contained in pollen extracts, b) a glycoprotein, c) a sulfhydryl group containing protein, d) a molecular weight about 30,000 as determined by SDS-polyacrylamide gel electrophoresis and e) possessing allergen activity.

45. The antibody of claim 44 wherein said antibody is a polyclonal antibody.

46. The antibody of claim 44 wherein said antibody is a monclonal antibody.


SEQUENCE LISTING (11 sequences)

SEQ ID NO: 1 - protein, 14 aa, Ragweed
  VARIANT 1,2: Xaa = Leucine or Isoleucine
XXSGISNTVYANPK

SEQ ID NO: 2 - protein, 9 aa, Ragweed
  VARIANT 6: Xaa= Leucine or Isoleucine
PTSFNXATK

SEQ ID NO: 3 - protein, 9 aa, Ragweed
  VARIANT 1: Xaa= Leucine or Isoleucine
XYGLVQFNR

SEQ ID NO: 4 - protein, 7 aa, Ragweed
  VARIANT 3: Xaa= Leucine or Isoleucine
FYXFSTK

SEQ ID NO: 5 - protein, 10 aa, Ragweed
  VARIANT 7,9: Xaa= Leucine or Isoleucine
FYATEVXDXD

SEQ ID NO: 6 - protein, 16 aa, Ragweed
LLDNLHQQTPPDGFGR

SEQ ID NO: 7 - protein, 13 aa, Ragweed
MYATEVLDLDGSK

SEQ ID NO: 8 - protein, 14 aa, Ragweed
YSDGNFFGAGLDHQ

SEQ ID NO: 9 - protein, 6 aa, Ragweed
LLNNMR

SEQ ID NO: 10 - protein, 8 aa, Ragweed
VEASAELR

SEQ ID NO: 11 - protein, 9 aa, Ragweed
LLSGLSDTV